(19) United States
(12) Patent Application Publication
(10) Pub. No.: US 2025/0361252 A1
(43) Pub. Date: Nov. 27, 2025

(54) APOMORPHINE PRODRUGS AND USES THEREOF

(71) Applicant: EVER Neuro Pharma GmbH, Unterach am Attersee (AT)

(72) Inventors: Maria-Lena ACHLEITNER, Unterach am Attersee (AT); Brigitta ELSÄSSER, Unterach am Attersee (AT); Johannes NIEDERHAUSER, Unterach am Attersee (AT); Zoltan TÖLGYESI, Unterach am Attersee (AT)

(73) Assignee: EVER Neuro Pharma GmbH, Unterach am Attersee (AT)

(21) Appl. No.: 18/872,223

(22) PCT Filed: Jun. 15, 2023
(86) PCT No.: PCT/EP2023/066146

§ 371 Date: Dec. 5, 2024

(30) Foreign Application Priority Data
Jun. 15, 2022 (EP) 22179225.2

(51) Int. Cl. C07F 9/576; A61K 31/675; A61K 45/06

(52) U.S. Cl. CPC C07F 9/5765; A61K 31/675; A61K 45/06

(57) ABSTRACT

The present invention relates to a compound of formula (I), which is a phosphate ester of apomorphine, or a pharmaceutically acceptable salt thereof. The apomorphine phosphate ester according to the invention exhibits remarkably advantageous properties as a therapeutic, including a favorable tolerability, an improved side effect profile, particularly a reduced occurrence of skin nodule formation and panniculitis when administered subcutaneously, as well as pharmacokinetic and metabolic properties rendering it particularly well-suited as an apomorphine prodrug. The invention further relates to the compound of formula (I) for use as a medicament, particularly for use in the treatment of Parkinson's disease.

The present invention relates to a compound of formula (I), which is a phosphate ester of apomorphine, or a pharmaceutically acceptable salt thereof. The apomorphine phosphate ester according to the invention exhibits remarkably advantageous properties as a therapeutic, including a favorable tolerability, an improved side effect profile, particularly a reduced occurrence of skin nodule formation and panniculitis when administered subcutaneously, as well as pharmacokinetic and metabolic properties rendering it particularly well-suited as an apomorphine prodrug. The invention further relates to the compound of formula (I) for use as a medicament, particularly for use in the treatment of Parkinson's disease.

Idiopathic Parkinson's disease is the second most common neurodegenerative disease worldwide. Although no available therapies alter the underlying neurodegenerative process, symptomatic therapies can improve patient quality of life. An estimated 7 to 10 million people are living worldwide with Parkinson's disease (Parkinson's Disease Foundation, Statistics on Parkinson). The average age of onset of Parkinson's disease (PD) is around 62 years. Most PD cases occur sporadically and are of unknown cause.

Clinically, Parkinson's disease is characterized by rest tremor, rigidity, bradykinesia and gait impairment, known as the “cardinal features” of the disease. Additional features include freezing of gait, postural instability, speech difficulty, autonomic disturbances, sensory alterations, mood disorders, sleep dysfunction, cognitive impairment and dementia, all known as non-dopaminergic features because they do not fully respond to dopaminergic therapy (Olanow and Schapira. Ann Neurol. 2013 September; 74(3):337-47). Pathologically, the hallmark features of PD are degeneration of pigmented mesostriatal dopaminergic neurons linking the substantia nigra (pars compacta) to the neostriatum (caudate nucleus and putamen). Other affected pigmented nuclei may include the locus ceruleus and dorsal motor nucleus of the vagus and intracytoplasmatic proteinaceous inclusions known as Lewy bodies. These are composed of misfolded and aggregated proteins. Mutations in α-syn promote misfolding of the protein and the formation of oligomers and aggregates thought to be involved in the cell death process (Olanow and Schapira. Ann Neurol. 2013 September; 74(3):337-47).

Early in the disease course, dopamine deficiency is the predominant neurochemical abnormality. In the progress of the disease, involvement of nondopaminergic brain regions results in levodopa-resistant motor and non-motor symptoms. Consequently, dopamine replacement therapy with levodopa is the gold standard for the initial treatment of Parkinson's disease. Despite the outstanding reputation of levodopa in the early stages of the disease, disabling fluctuations in motor response and dyskinesias constitute the major threat during long-term therapy.

Motor fluctuations in motor performance eventually develop in >50% of patients with Parkinson's disease treated with oral levodopa for >5 years. In addition, many patients also experience other unpleasant “off-period” phenomena, including mood swings, delusions, anxiety and painful dystonia that coincide with their motor state (Cantello R, et al. Neurol Neurosurg Psychiatry. 1986 October; 49(10):1182-90; Hardie R J, et al. Brain. 1984 June; 107(Pt 2):487-506; Nissenbaum H, et al. Psychol Med. 1987 November; 17(4):899-904; Quinn N P, et al. Lancet. 1986; 327(8494):1366-1369).

Initially, these response oscillations exhibit a predictable pattern related to the timing of levodopa intake (“wearing-off” phenomenon) and can be managed by shortening the levodopa-dose intervals, an addition of a monoamine oxidase (MAO)-B inhibitor (like selegiline/deprenyl) or dopamine receptor agonists and the administration of controlled-release preparations of levodopa or catechol-O-methyl-transferase inhibitors (e.g., entacapone). However, in the advanced stages of the disease, patients experience complex and unpredictable motor oscillations referred to as “on-off” phenomenon, which are refractory to these conventional therapeutic strategies (Marsden C D, et al. Lancet. 1977 Feb. 12; 1(8007):345-9).

Eventually, the clinical response closely reflects peripheral L-dopa pharmacokinetics, characterized by a plasma half-life of 1-1.5 hours. While the peripheral pharmacokinetics of L-dopa remain unchanged throughout the course of the illness, pre-synaptic nigrostriatal nerve terminals gradually lose their ability to store dopamine. However, evidence exists for a far more complex basis of the development of motor complications that are likely to be related to long-term unphysiological, pulsatile stimulation of the dopamine receptors and involve changes in striatal gene expression and subsequently in altered firing patterns of the basal ganglia.

Motor complications are divided into motor fluctuations and dyskinesia. With advancing PD patients may begin to fluctuate in motor performance, that is to experience a “wearing-off” (end-of-dose) effect because the motor improvement after a dose of levodopa becomes reduced in duration and parkinsonism reappears. A minority of patients may experience diphasic dyskinesia, in which they exhibit dyskinesia at the beginning of turning “on” and/or at the beginning of turning “off”, but have different and less severe or absent dyskinesia at the time of peak levodopa effect. Eventually patients may experience rapid and unpredictable fluctuations between “on” and “off” periods known as the “on-off” phenomenon.

The management of motor fluctuations aims to prolong the effect of individual L-dopa doses by adding adjuvant drugs, such as catechol-O-methyl transferase (COMT) and monoamine oxidase B (MAO-B) inhibitors, as well as changing the intervals between intakes and advising patients to avoid taking L-dopa with meals. Also transdermal dopamine agonists are added to the drug regime or their dose is increased. In some patients, attempts to adjust oral and transdermal medication in the presence of disabling fluctuations and dyskinesias fail after months or years. Further options, which include deep-brain stimulation, a pump system that delivers L-dopa to the jejunum via a gastric tube and the dopamine agonist apomorphine, which is delivered subcutaneously either intermittently or continuously, are therapy options for late-stage PD patients suffering from motor fluctuations.

Apomorphine is the oldest dopamine agonist used in clinical practice and is indicated for the treatment of motor symptoms associated with late-stage Parkinson's disease, specifically for the acute, intermittent treatment of hypomobility, “off” episodes (“end-of-dose wearing off” and unpredictable “on/off” episodes) associated with advanced Parkinson's disease, and as adjunct/supplemental therapy to standard levodopa therapy. It was first applied in PD patients in 1951, but interest waned when oral L-dopa was introduced. As the long-term complications associated with L-dopa therapy became recognized, and the antiemetic domperidone (apomorphine leads to severe emesis), which in doses of 10-30 mg tds for 72 hours before apomorphine can prevent most peripheral dopaminergic side effects, became available, apomorphine was investigated further.

Apomorphine is not effective orally due to extensive first-pass metabolism in the liver. The precise mechanism of action of apomorphine as a treatment for Parkinson's disease is unknown, although it is believed to be due to stimulation of post-synaptic D2 receptors within the caudate putamen, a brain structure which supports motor function.

There are currently two distinct methods of administering apomorphine: subcutaneous bolus doses and continuous infusion. When injected subcutaneously, its bioavailability reaches nearly 100% and injections can be effective in rapidly resolving off states in patients with motor fluctuations. When given as a single dose, symptom relief is equivalent to oral L-dopa, with a considerably faster onset (five to 15 minutes) and shorter duration (mean 40 minutes) of effect. Intermittent apomorphine injections may be used to reduce off time in people with PD with severe motor complications. Continuous subcutaneous infusions of apomorphine may be used to reduce “off” time and dyskinesia in people with PD with severe motor complications. Subcutaneous infusions of apomorphine are appropriate for PD patients with so many off periods that repeated bolus injections are inappropriate.

Apomorphine, synthesized from morphine by heating with HCl as a catechol derivative, is known to be sensitive for oxidation. Under the influence of oxygen, solutions of apomorphine turn into green color, indicating the formation of oxidation products with “quinone-background” (Neef C, et al. Clin Pharmacokinet. 1999. 37(3):257-71).

Electrochemical oxidation experiments have shown that the oxidative apomorphine degradation is pH-dependent.

Degradation products increase with increasing pH, leading to a spontaneous autooxidation at neutral pH (Garrido J M, et al. Bioelectrochemistry. 2002. 55(1-2):113-4).

Water solubility of apomorphine is in pure water at acidic pH 20 mg/ml, whereas in NaCl solution solubility decreases to lower 10 mg/ml. The pKₐ values of the apomorphine-hydrochloride are 7.2 and 8.9, respectively. UV absorption maxima in 0.1 mM HCl solution is at 273 nm and a small shoulder at 305 nm (Muhtadi F J, et al. Analytical Profiles of Drug Substances. 1991. 20:121-171; also confirmed by own data).

Many routes of administration of apomorphine have been tried in clinical approaches resulting in therapeutic effective application as subcutaneous, sublingual, nasal or rectal administration. Currently only subcutaneous formulations are used in clinical routine (Neef C, et al. Clin Pharmacokinet. 1999. 37(3):257-71).

When administered subcutaneously, apomorphine is known to induce adverse effects at the site of administration, such as skin changes, irritabilities at injection sites, and subcutaneous nodules or panniculitis (inflammation of the subcutaneous adipose tissue).

Apomorphine formulations available on the market are stabilized by low pH (3-4) and sodium metabisulfite as antioxidative substance. In some cases, sulfites can induce allergic reactions in some patients. In addition, sodium metabisulfite tends to react irreversibly with carbon-oxygen double bonds found in aldehydes and ketones. This is evaluated, e.g., for epinephrine, which similarly to apomorphine contains two aromatic hydroxyl bound groups in ortho position leading to quinone formation during oxidation (Gupta P K, et al. (eds.). Injectable drug development: techniques to reduce pain and irritation. Taylor and Francis Group. 1999. 409).

Histological data have shown that apomorphine induces melanin-positive pigmentation in the s.c. area (Loewe R, et al. Hautarzt. 2003. 54:58-63). Additional data support the described nodule formation at the injection site as a panniculitis with eosinophile and neutrophile infiltration without increased IgE infiltration (Acland K M, et al. Br J Dermatol. 1998. 138(3):480-2). These irritations lead to a termination of therapy in 70% of the patients receiving apomorphine by subcutaneous infusion within one year.

Nodule formation induced by subcutaneous apomorphine application is one of the most frequently described side effects in injection or infusion therapy with apomorphine. In a meta-analysis the incidence of nodule formation was determined with 70% in subcutaneous apomorphine infusion therapy (Deleu D, et al. Drugs Aging. 2004. 21(11):687-709). This side effect is also described in all summaries of product characteristics (SmPCs) of approved apomorphine solutions.

However, even if there are descriptions of symptoms, a clear root cause of nodule formation is still missing. For example, Edwards et al. recently noted that “Few studies have been conducted on the formation of apomorphine nodules and consequently, little is known about their aetiology or natural history.” (Edwards H, et al. Ultrasound. 2008. 16(3):155-9). Allergic reactions, hygienic reasons, effects induced by the excipients (EDTA or sodium metabisulfite) and dopamine toxicity have been discussed as root cause of nodule formation, without clear evidence for any of these hypotheses (Acland K M, et al. Br J Dermatol. 1998. 138(3):480-2; Boyle A, et al. CNS Drugs. 2015. 29:83-9; Dadban A, et al. Annales de Dermatologie et de Venersologie. 2010. 137(11):730-5; Deleu D, et al. Drugs Aging. 2004. 21(11):687-709; Edwards H, et al. Ultrasound. 2008. 16(3):155-9; Ganesaligam J, et al. Movement Disorders. 2011. 26(12):2182; Henriksen T. Neurodegen. Dis. Manage. 2014. 4(3):271-82; Hughes A J, et al. Movement Disorders. 1993. 8(2):165-70; Loewe R, et al. Hautarzt. 2003. 54:58-63; Martinez-Martin P, et al. Movement Disorder. 2015. 30(14):510-6; Neef C, et al. Clin Pharmacokinet. 1999. 37(3):257-71; Rosei M A, et al. Biochemistry and Molecular Biology International. 1995. 35(6):1253-9).

Several trials were made in the past to explain the neuro-toxic effect of apomorphine on a cellular basis. The results of these investigations described in the following can be of help for understanding the mechanism behind the described side effects of apomorphine-formulations at the subcutaneous injection site. In cytotoxicity studies it could be demonstrated that apomorphine has an anti-proliferative effect and induces apoptosis on the CHO-K1 cell line (Maggio R, et al. Neurotox Res. 2000. 1(4):285-97; Pardini C, et al. Neuropharmacology. 2003. 45(2):182-9). Other studies on rat glioma C6 cells and rat cultured neurons showed that apomorphine promotes the loss of cell membrane integrity, degeneration of cytoplasmic organelles (especially mitochondria), DNA fragmentation and necrosis in vitro most likely through the formation of oxidative degradation products of apomorphine (quinones) (El-Bacha R S, et al.

Neuroscience Letters. 1999. 263:25-8; dos Santos El-Bacha R, et al. Biochem Pharmacol. 2001. 61(1):73-85).

Further it was shown that apomorphine exerts an anti-proliferative effect on several tumor cell lines (Kondo Y, et al. J Pharmacobiodyn. 1990. 13(7):426-31; Schrell U M, et al. J Clin Endocrinol Metab. 1990. 71(6):1669-71).

Also a genotoxic activity of apomorphine was demonstrated in vitro and in vivo and might be related to its ability to intercalate into DNA or to its pro-oxidant effects or generation of superoxide radicals during autoxidation, hence promoting frameshift mutations and inducing oxidative mutagenesis. These mutagenic and clastogenic effects are most likely due to quinone products formed by oxidation of apomorphine (reviewed in: Picada J N, et al. Brazilian journal of medical and biological research. 2005. 38:477-86; Picada J N, et al. Mutat Res. 2003. 539(1-2):29-41; Picada J N, et al. Brain Res Mol Brain Res. 2003. 114(1):80-5) as the more aromatic and planar structure of quinone products favor the intercalation into DNA (Cheng H, et al. Analytical Chemistry. 1979. 51(13):2243-6; Kalyanaraman B. Methods Enzymol. 1990. 186:333-43). It is known that quinones in general are metabolically active intermediates with a toxicological potential leading to several toxic effects in vivo (Garrido J M, et al. J Chem Soc, Perkin Trans 2. 2002. 10:1713-7). Apomorphine was not evidently genotoxic in the in vivo studies performed, however, genotoxic effects of apomorphine and/or its oxidative products as well as its ability to intercalate into DNA can lead to cell death and might be an explanation for the cytotoxic and anti-proliferative effects of apomorphine observed in the studies mentioned above.

Apomorphine can undergo spontaneous autooxidation in neutral and alkaline solutions (Kaul P N. J Pharm Sci. 1961. 50:266-7), which reflects the physiological environment of the subcutaneous tissue and reactive metabolites, such as quinones and reactive oxygen species (ROS) may be produced during this oxidative mechanism. Yet, even in acidic solutions a significant oxidative degradation of apomorphine occurs in the absence of antioxidants, resulting in a green coloration of the solution within a single day. At pH<7 the main degradation product is oxoapomorphine, whereas at pH >7 apomorphine-paraquinone is the main degradation product (Udvardy A, et al. Journal of molecular structure. 2011. 1002(1):37-44). Therefore the data from in vitro studies, mentioned above, fit into commonly observed adverse effects of apomorphine at the site of administration, such as skin changes, irritabilities at injection sites and subcutaneous nodules and panniculitis, as oxidation of apomorphine subcutaneously and the generation of oxidative products of apomorphine, such as quinones or semi-quinones, may constitute the mechanisms leading to the loss of cellular integrity and cell death (necrosis) that subsequently trigger the onset of the observed inflammatory processes and nodule formation in the surrounding area of the injection site.

Various attempts have been made to improve the tolerability, stability and/or the pharmacokinetic properties of apomorphine. In particular, specific formulations of apomorphine have been proposed, e.g., in EP-A-2545905, U.S. Pat. Nos. 5,939,094, 6,121,276, 8,772,309, WO 99/66916, WO 02/100377, WO 2009/019463, WO 2009/056851, WO 2013/007381, WO 2013/183055, and WO 2017/055337. Moreover, certain prodrugs of apomorphine have also been proposed but have not resulted in any approved medicinal products; see, e.g., WO 2003/080074; WO 2005/041966; Borgman R J et al., J Med Chem, 1976, 19(5):717-19, doi: 10.1021/jm00227a026; Liu K S et al., EurJPharm Biopharm, 2011, 78(3):422-31, doi: 10.1016/j.ejpb.2011.01.024; Borkar N et al., EurJPharm Biopharm, 2015, 89:216-23, doi: 10.1016/j.ejpb.2014.12.014; and Borkar N et al., Asian J Pharm Sci, 2018, 13(6):507-517, doi: 10.1016/j.ajps.2017.11.004.

WO 2019/101917 discloses prodrugs of apomorphine in the form of a sulfate ester with either one or both of the 10- and 11-hydroxy groups modified. These compounds are disclosed as reference examples that are considered unsuitable for use as orally bioavailable prodrugs.

Park Hyejin et al. (ACS Medicinal Chemistry Letters, vol. 11, no. 3, 12 Mar. 2020, pages 385-392) discloses several types of apomorphine prodrugs, including the di-acetate derivative, the di-MOM ether derivative and the methylene acetal derivative.

Thus, there is still a strong and unmet need for novel therapeutic approaches for providing apomorphine with improved safety and tolerability and an improved side effect profile.

The present invention addresses this need and provides a novel prodrug of apomorphine that can be administered subcutaneously with highly advantageous safety and tolerability and a particularly beneficial side effect profile, which allows to prevent or reduce the occurrence of inflammatory reactions, nodule formation and panniculitis in the subcutaneous tissue at the site of administration.

Thus, it has surprisingly been found in the context of the present invention that apomorphine phosphate esters, particularly the compounds of formula (I) and their pharmaceutically acceptable salts, are advantageously water-soluble in comparison to apomorphine or its pharmaceutically acceptable salts. The present inventors have furthermore surprisingly found that the compounds of formula (I) and their pharmaceutically acceptable salts are physiologically stable, particularly in human blood and in the subcutaneous tissue, but are readily metabolized by human hepatocytes to apomorphine (as also demonstrated in Example 6), which makes them particularly well suited as prodrugs. The compounds of formula (I) have thus been found to be highly advantageous as they show superior tolerability, improved stability both under storage conditions and under physiological conditions in blood and in the subcutaneous tissue, as well as an improved side effect profile, in particular a considerably reduced occurrence of inflammation and panniculitis at the site of subcutaneous administration, which greatly facilitates patient acceptance and compliance. This is further confirmed by the data shown in Example 7, which demonstrates a reduced development of nodules by pigs treated with the apomorphine prodrug of the invention when compared to a commercially available state-of-the-art formulation of apomorphine.

Accordingly, in a first aspect, the present invention provides a compound of formula (I) or a pharmaceutically acceptable salt thereof:

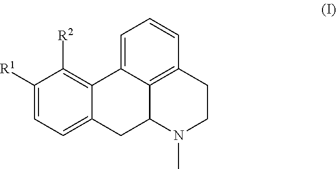

In formula (I), the groups R¹ and R² are each independently —OH or —O—P(═O)(—OH)(—OH), provided that at least one of R¹ and R² is —O—P(═O)(—OH)(—OH).

In a second aspect, the invention relates to a pharmaceutical composition comprising the compound according to the first aspect, and a pharmaceutically acceptable excipient.

In a third aspect, the invention relates to the compound according to the first aspect or to the pharmaceutical composition according to the second aspect for use as a medicament (or for use in therapy). In accordance with this third aspect, the invention also relates to the use of the compound according to the first aspect in the preparation of a medicament.

In a fourth aspect, the present invention refers to the compound according to the first aspect or the pharmaceutical composition according to the second aspect for use in the treatment of a neurodegenerative disease/disorder. In this fourth aspect, the invention also relates to the use of the compound according to the first aspect in the preparation of a medicament for the treatment of a neurodegenerative disease/disorder. The invention further provides a method of treating a neurodegenerative disease/disorder, the method comprising administering a therapeutically effective amount of the compound according to the first aspect or the pharmaceutical composition according to the second aspect to a subject (e.g., a human) in need thereof. In accordance with the fourth aspect of the invention, the neurodegenerative disease/disorder is preferably selected from Parkinson's disease, Alzheimer's disease, Huntington's disease, neuroleptic malignant syndrome, dystonia, and schizophrenia (e.g., chronic schizophrenia), and is more preferably Parkinson's disease.

The invention is particularly concerned with the treatment of Parkinson's disease using the compound according to the first aspect or the pharmaceutical composition according to the second aspect as described and defined herein. Accordingly, in a fifth aspect, the present invention relates to the compound according to the first aspect or the pharmaceutical composition according to the second aspect for use in the treatment of Parkinson's disease (e.g., idiopathic Parkinson's disease, acquired Parkinson's disease, or hereditary Parkinson's disease), preferably in a human. In this aspect, the invention further refers to the use of the compound according to the first aspect in the preparation of a medicament for the treatment of Parkinson's disease. In the fifth aspect, the invention likewise provides a method of treating Parkinson's disease, the method comprising administering a therapeutically effective amount of the compound according to the first aspect to a subject (e.g., a human) in need thereof.

The compound according to the first aspect of the invention or the pharmaceutical composition according to the second aspect of the invention can also be used as a rescue treatment of subjects suffering from Parkinson's disease. In particular, the compound or the pharmaceutical composition comprising the same can be used as an acute treatment of parkinsonian subjects who have been receiving a medication (particularly a chronic medication) different from apomorphine and who are suffering from an acute off-period. The compound or the pharmaceutical composition can thus be administered on demand when a subject receiving a different treatment of Parkinson's disease (e.g., levodopa) experiences motor fluctuations between regular treatment doses (e.g., between regular doses of levodopa). The compound or the pharmaceutical composition may also be administered to subjects who suffer from off-periods of more than about 30 min.

Thus, in a sixth aspect, the present invention relates to the compound according to the first aspect or the pharmaceutical composition according to the second aspect for use in the treatment of refractory motor fluctuations/oscillations in Parkinson's disease, off-periods in Parkinson's disease, refractory off-periods in Parkinson's disease, dyskinesia (particularly peak-dose dyskinesia) in Parkinson's disease, or akinesia in Parkinson's disease. In this sixth aspect, the invention further refers to the use of the compound according to the first aspect in the preparation of a medicament for the treatment of refractory motor fluctuations/oscillations in Parkinson's disease, off-periods in Parkinson's disease, refractory off-periods in Parkinson's disease, dyskinesia in Parkinson's disease, or akinesia in Parkinson's disease. The invention also relates to a method of treating refractory motor fluctuations/oscillations in Parkinson's disease, off-periods in Parkinson's disease, refractory off-periods in Parkinson's disease, dyskinesia in Parkinson's disease, or akinesia in Parkinson's disease, the method comprising administering a therapeutically effective amount of the compound according to the first aspect or the pharmaceutical composition according to the second aspect to a subject (e.g., a human) in need thereof.

In a seventh aspect, the invention provides the compound according to the first aspect or the pharmaceutical composition according to the second aspect for use in the treatment of sexual dysfunction or impotence (including male or female sexual dysfunction, particularly male erectile dysfunction), preferably in a human subject. In accordance with this seventh aspect, the invention thus relates, in particular, to the compound according to the first aspect or the pharmaceutical composition according to the second aspect for use in the treatment of male erectile dysfunction in a human subject. In this seventh aspect, the invention likewise refers to the use of the compound according to the first aspect in the preparation of a medicament for the treatment of sexual dysfunction or impotence, particularly for the treatment of male erectile dysfunction in a human subject. The invention also provides a method of treating sexual dysfunction or impotence, the method comprising administering a therapeutically effective amount of the compound according to the first aspect or the pharmaceutical composition according to the second aspect to a subject (e.g., a human) in need thereof. In particular, the invention provides a method of treating male erectile dysfunction, the method comprising administering a therapeutically effective amount of the compound according to the first aspect or the pharmaceutical composition according to the second aspect to a human subject in need thereof.

In an eighth aspect, the invention is directed to the compound according to the first aspect or the pharmaceutical composition according to the second aspect for use in the treatment of restless legs syndrome. In accordance with this eighth aspect, the invention also provides the use of the compound according to the first aspect in the preparation of a medicament for the treatment of restless legs syndrome. Likewise, the invention provides a method of treating restless legs syndrome, the method comprising administering a therapeutically effective amount of the compound according to the first aspect or the pharmaceutical composition according to the second aspect to a subject (e.g., a human) in need thereof.

In a ninth aspect, the present invention relates to the compound according to the first aspect or the pharmaceutical composition according to the second aspect for use in preventing, reducing or ameliorating panniculitis associated with the subcutaneous administration of apomorphine, wherein the compound or the pharmaceutical composition is to be administered subcutaneously. In this aspect, the invention also relates to the compound according to the first aspect or the pharmaceutical composition according to the second aspect for use in preventing, reducing or ameliorating the formation of subcutaneous nodules associated with the subcutaneous administration of apomorphine, wherein the compound or the pharmaceutical composition is to be administered subcutaneously. The invention further relates to the compound according to the first aspect or the pharmaceutical composition according to the second aspect for use in preventing, reducing or ameliorating inflammation and/or irritation of the skin associated with the subcutaneous administration of apomorphine, wherein the compound or the pharmaceutical composition is to be administered subcutaneously. In accordance with the ninth aspect, the invention furthermore refers to (i) the use of the compound according to the first aspect in the preparation of a medicament for preventing, reducing or ameliorating panniculitis associated with the subcutaneous administration of apomorphine, wherein the medicament is to be administered subcutaneously, (ii) the use of the compound according to the first aspect in the preparation of a medicament for preventing, reducing or ameliorating the formation of subcutaneous nodules associated with the subcutaneous administration of apomorphine, wherein the medicament is to be administered subcutaneously, and also (iii) the use of the compound according to the first aspect in the preparation of a medicament for preventing, reducing or ameliorating inflammation and/or irritation of the skin associated with the subcutaneous administration of apomorphine, wherein the medicament is to be administered subcutaneously. In the ninth aspect, the invention likewise refers to (i) a method of preventing, reducing or ameliorating panniculitis associated with the subcutaneous administration of apomorphine, the method comprising subcutaneously administering a therapeutically effective amount of the compound according to the first aspect or the pharmaceutical composition according to the second aspect to a subject (e.g., a human) in need thereof, (ii) a method of preventing, reducing or ameliorating the formation of subcutaneous nodules associated with the subcutaneous administration of apomorphine, the method comprising subcutaneously administering a therapeutically effective amount of the compound according to the first aspect or the pharmaceutical composition according to the second aspect to a subject (e.g., a human) in need thereof, and (iii) a method of preventing, reducing or ameliorating inflammation and/or irritation of the skin associated with the subcutaneous administration of apomorphine, the method comprising subcutaneously administering a therapeutically effective amount of the compound according to the first aspect or the pharmaceutical composition of the second aspect to a subject (e.g., a human) in need thereof.

The invention is also described by the following illustrative figures:

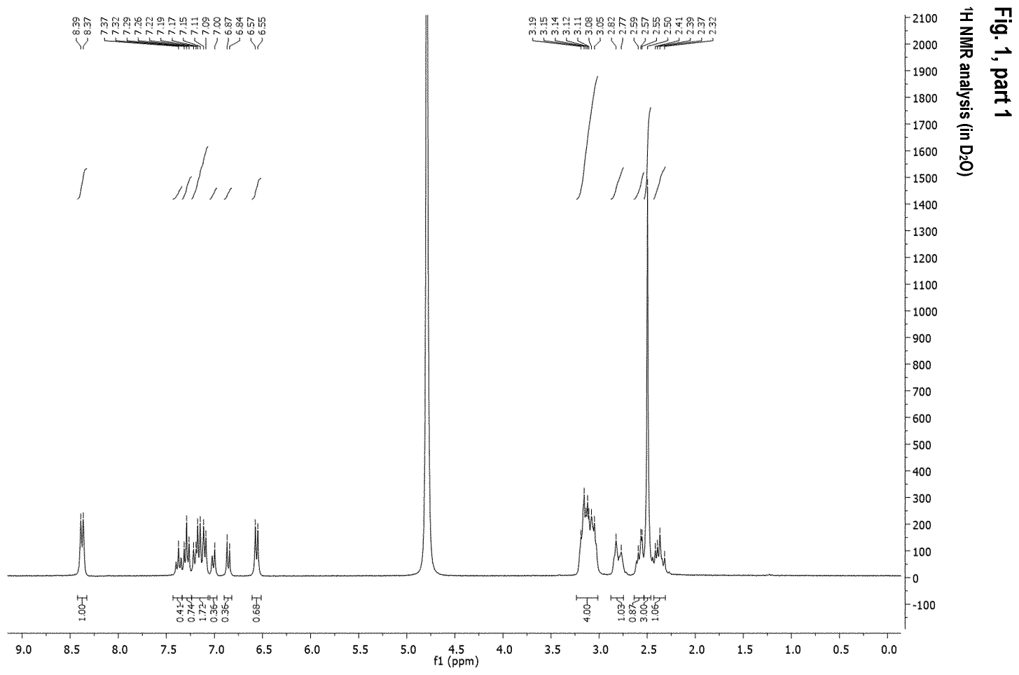

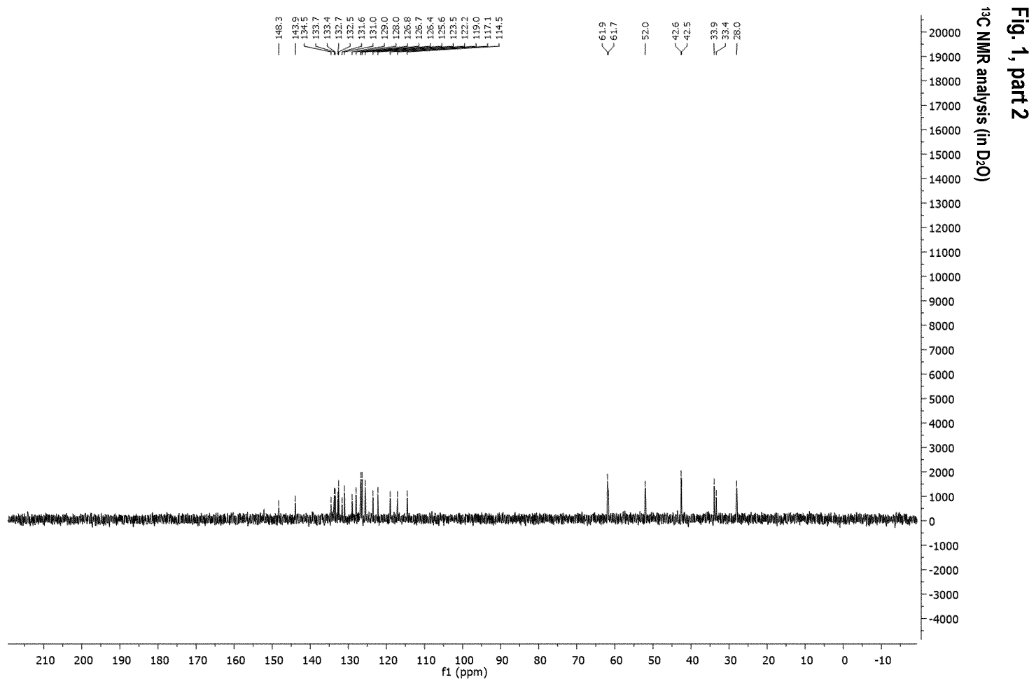

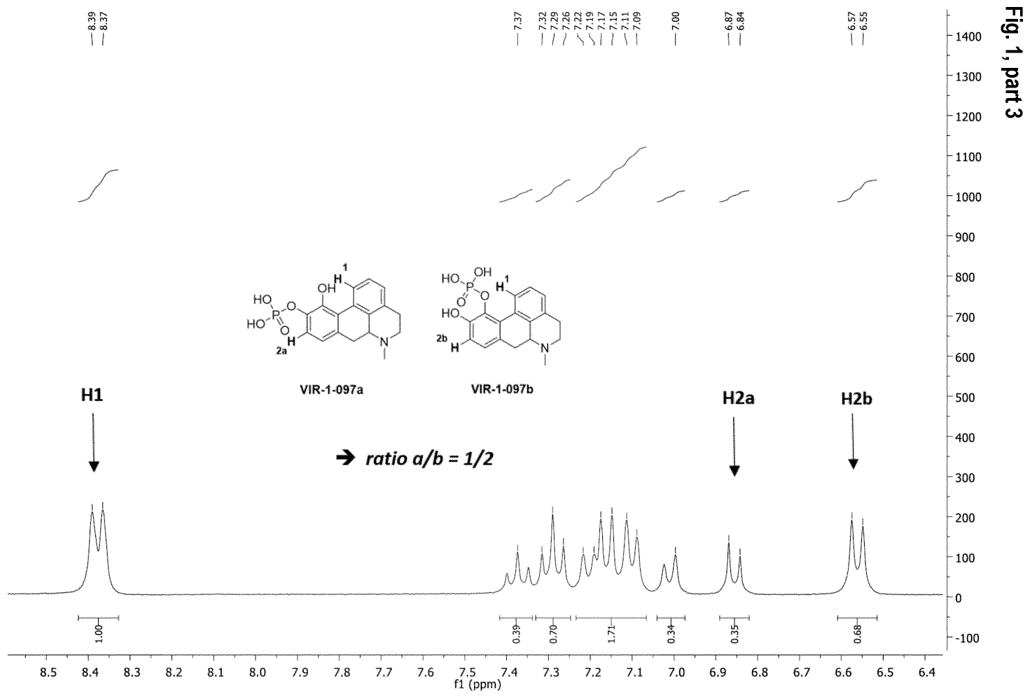

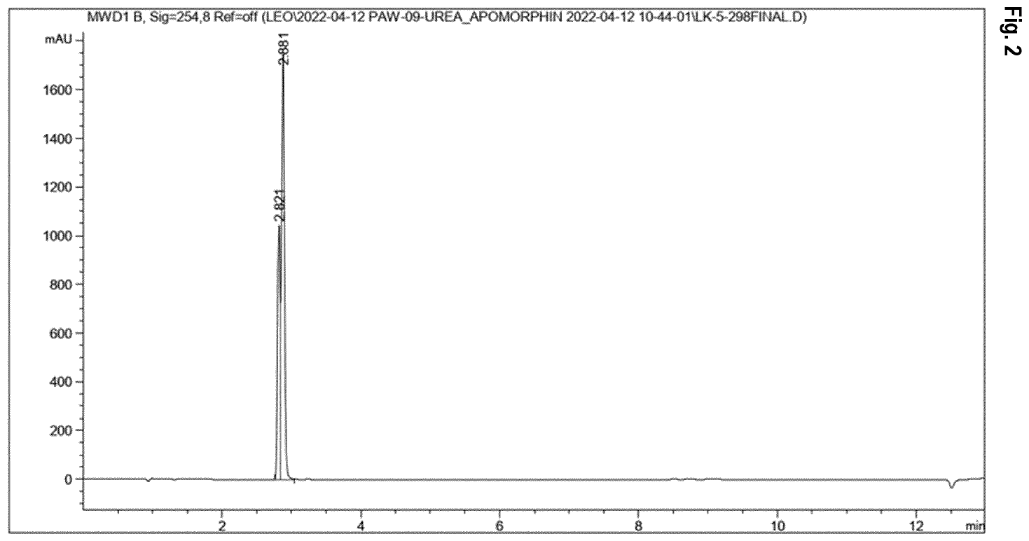

FIG. 1: Part 1 and part 2 show ¹H and ¹³C NMR spectra obtained in D₂0 for apomorphine monophosphate isomer mixture obtained in Example 1, respectively. Part 3 shows the basis for the calculation of the molar ratio of two isomers in the obtained mixture in Example 1.

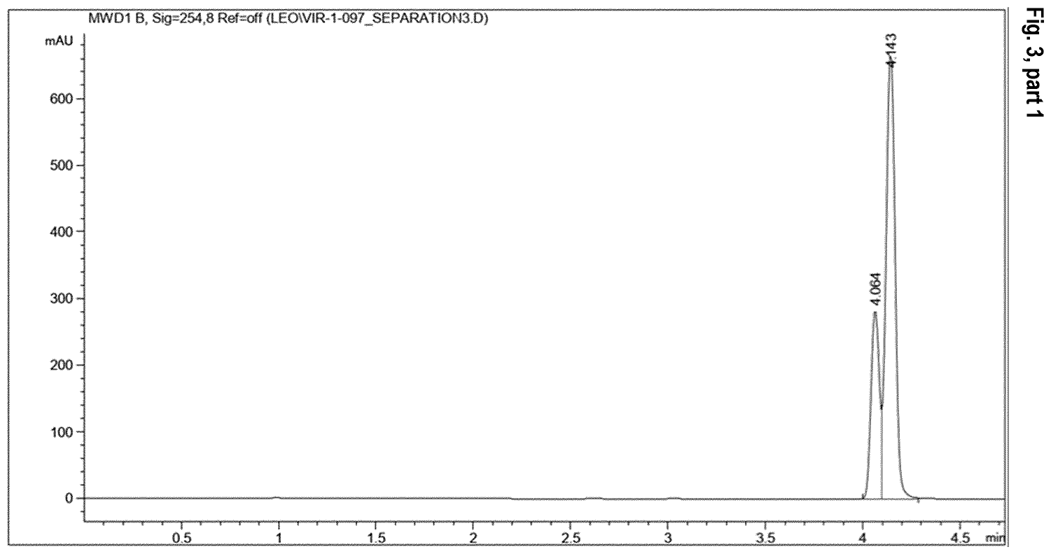

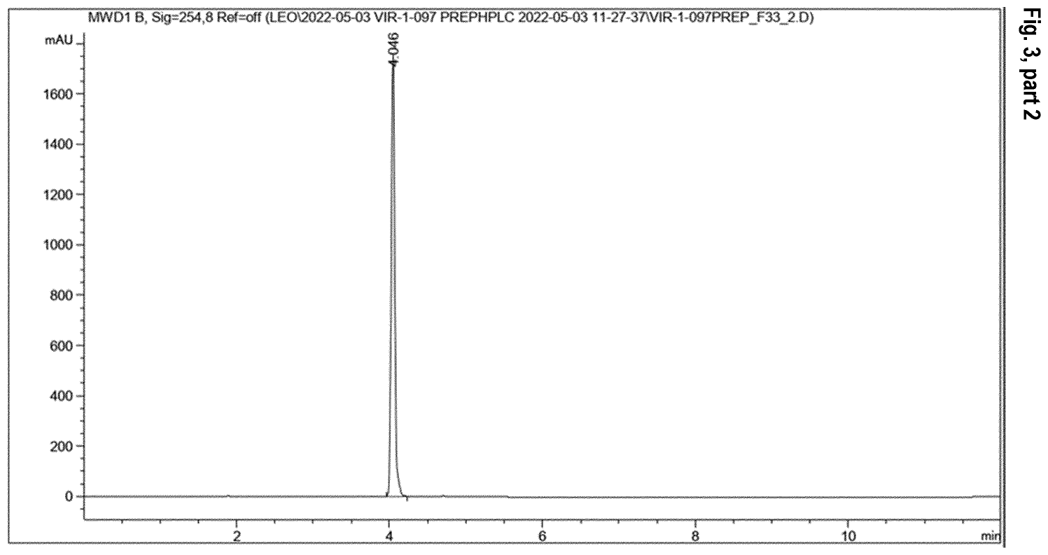

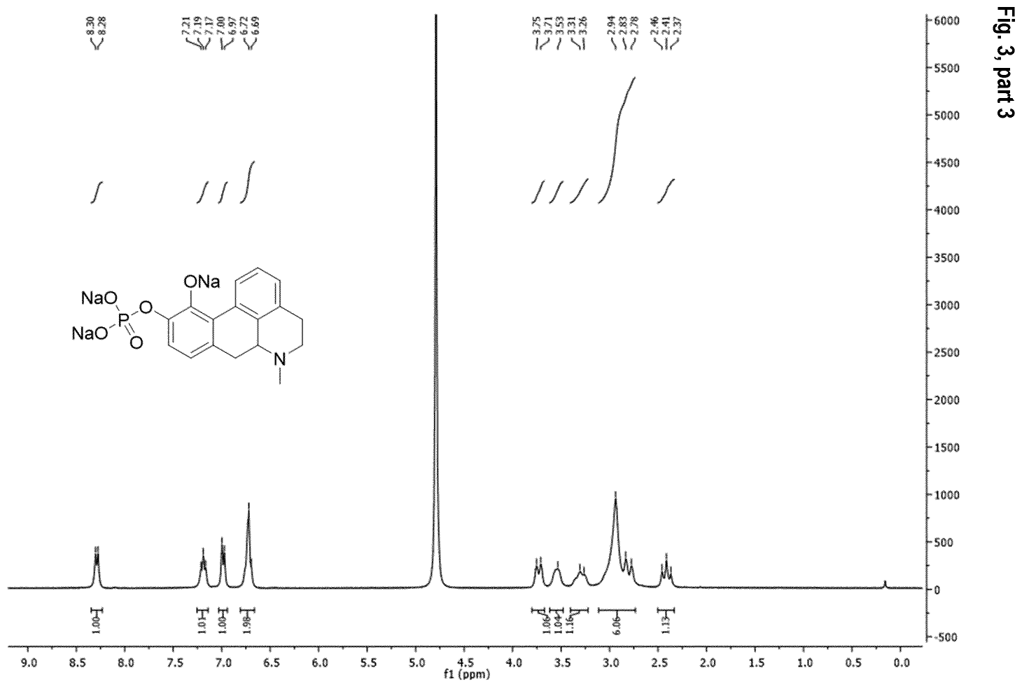

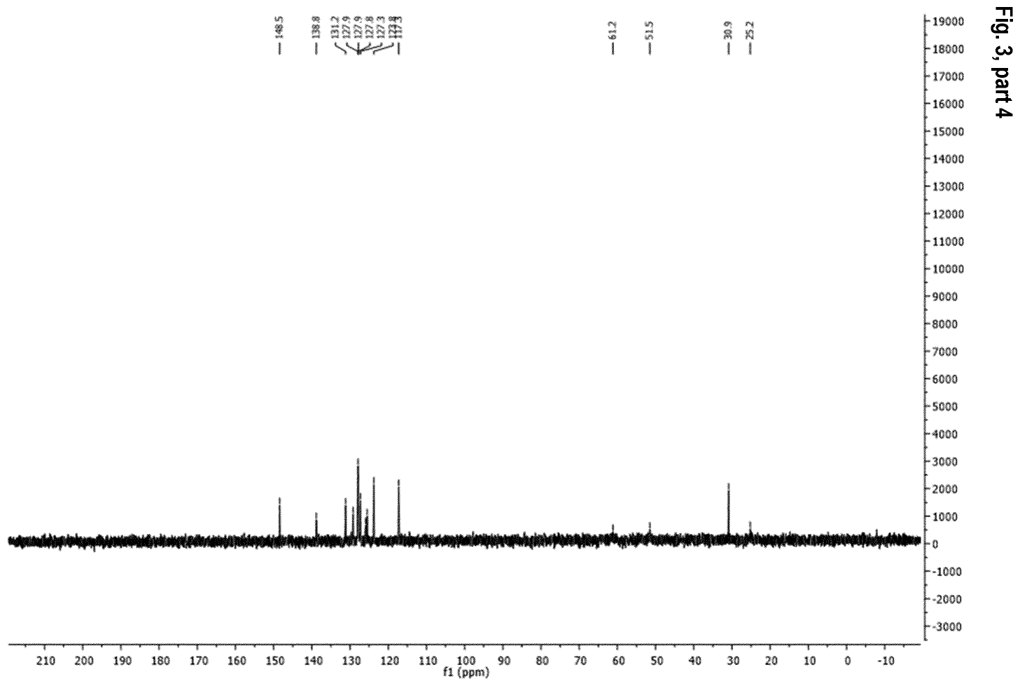

FIG. 2: HPLC-MS measurements obtained for the mixture obtained in Example 1. Both constitutional isomers of apomorphine phosphate are present (ratio˜2/1), m/z=348 [M(free acid)+H+].

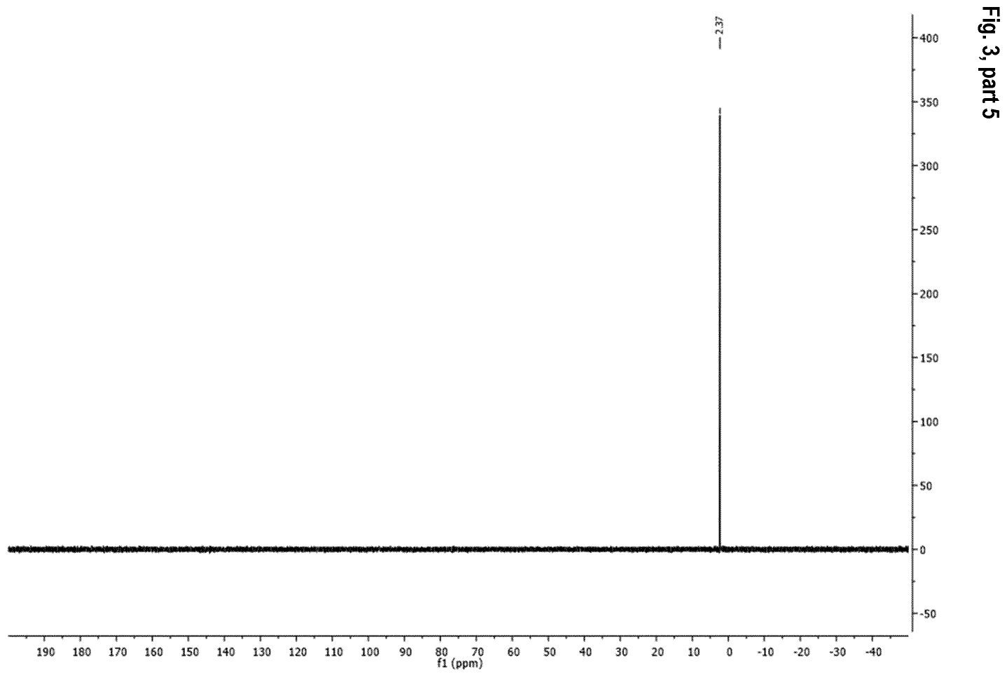

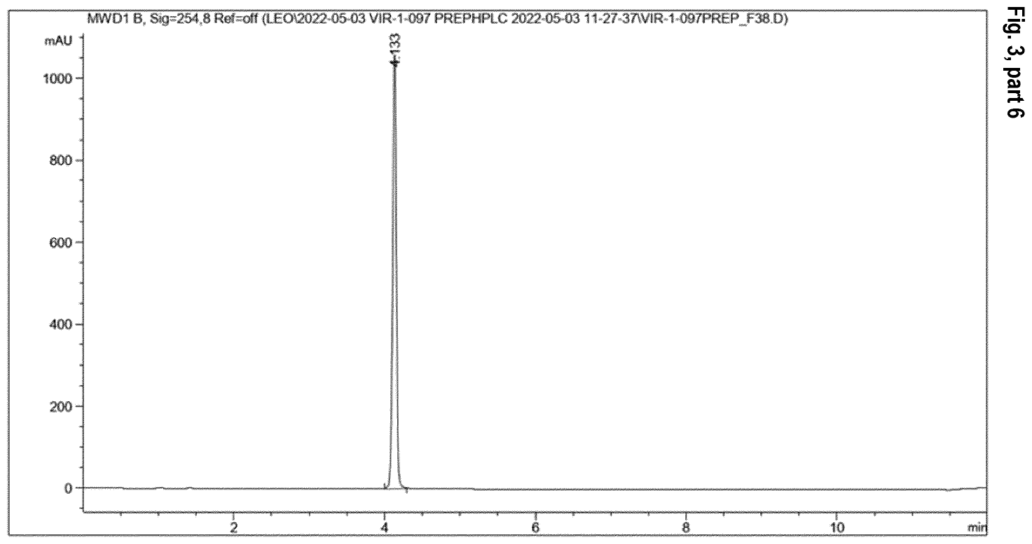

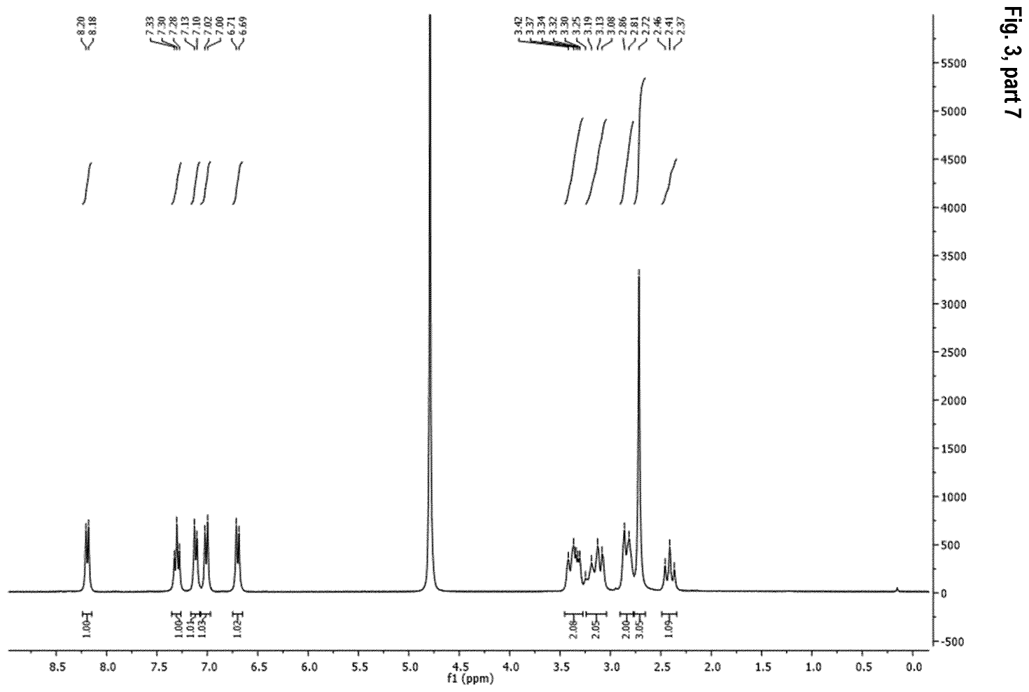

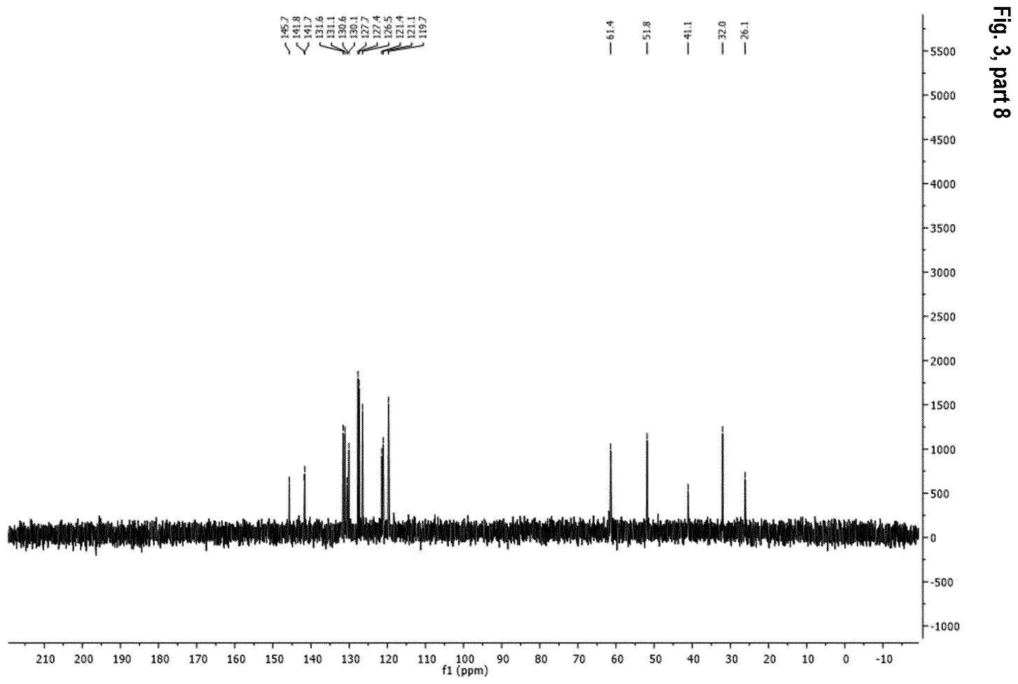

FIG. 3: Part 1—HPLC-MS analysis of apomorphine monophosphate isomer mixture subjected to separation in Example 2. Part 2—HPLC-MS analysis of the isomer contained in the first separated peak. Part 3-1H NMR analysis of the isomer contained in the first isolated peak. Part 4—¹³C NMR analysis of the isomer contained in the first isolated peak. Part 5—³¹P NMR analysis of the isomer contained in the first isolated peak. Part 6—HPLC-MS analysis of the isomer contained in the second separated peak. Part 7—¹H NMR analysis of the isomer contained in the second isolated peak. Part 8—¹³C NMR analysis of the isomer contained in the second isolated peak. Part 9—³¹P NMR analysis of the isomer contained in the second isolated peak.

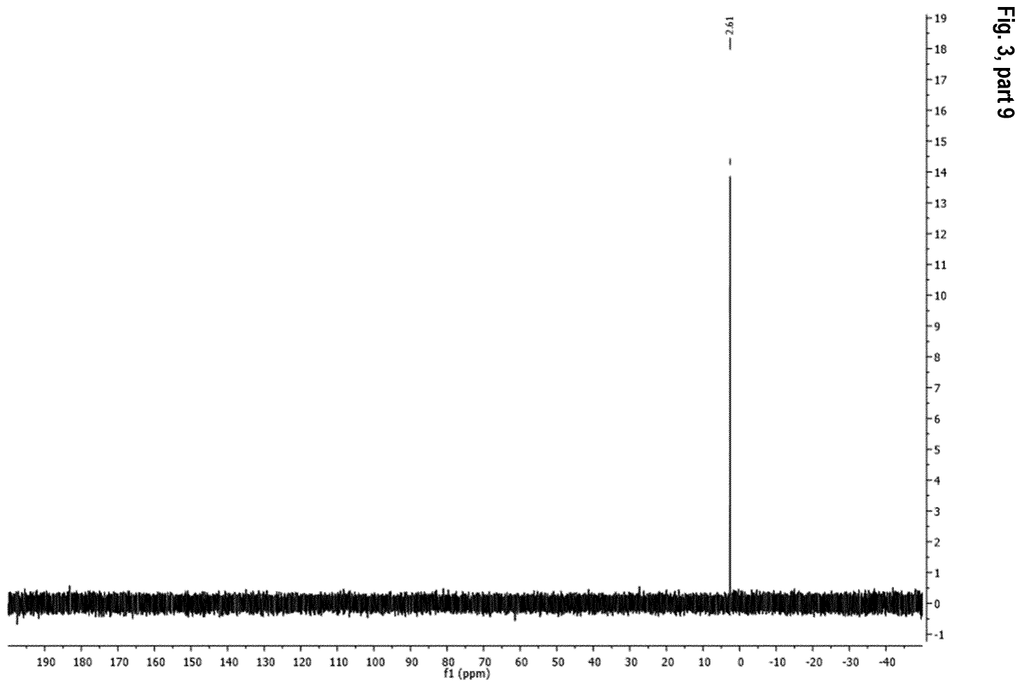

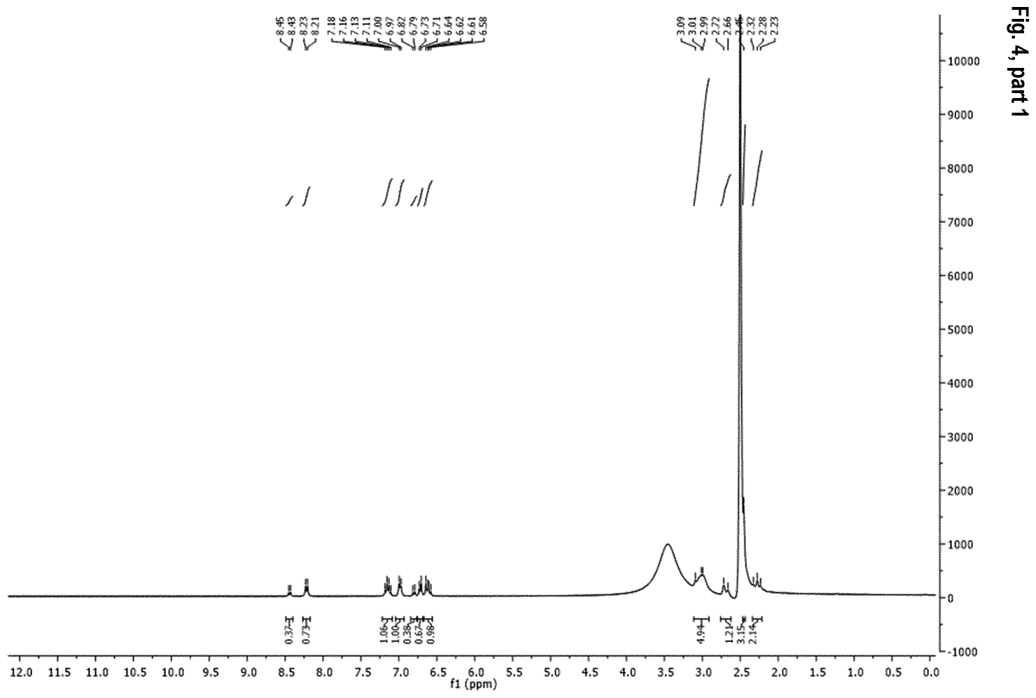

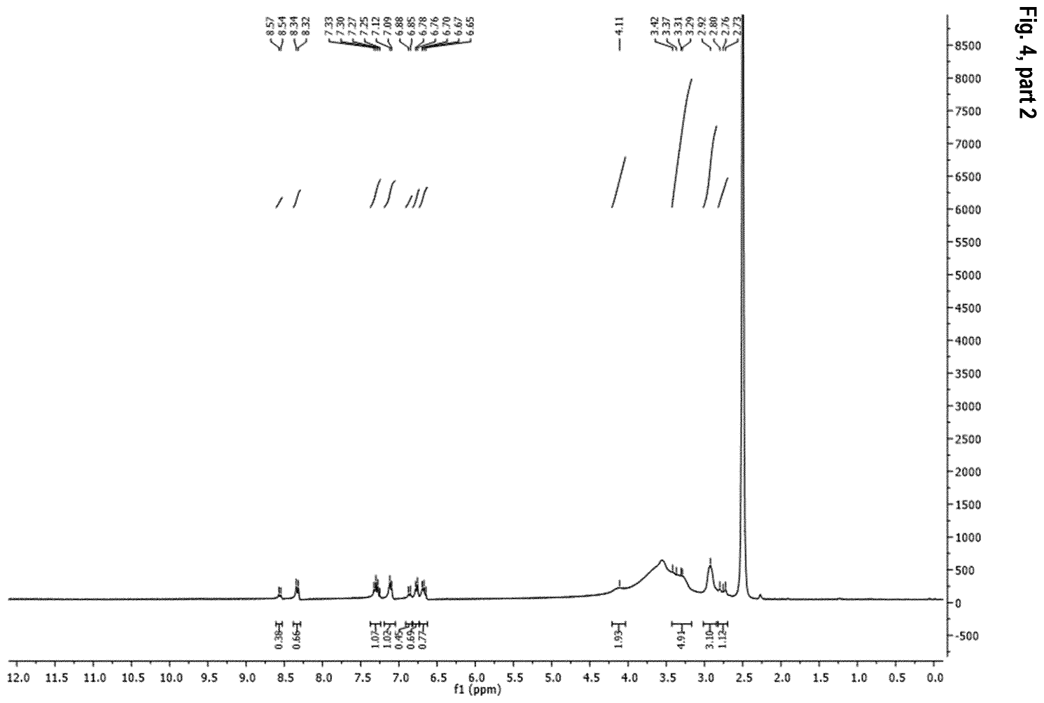

FIG. 4: Part 1—¹H NMR analysis of the isomer mixture obtained in Example 1 upon adjustment of pH to 8. Part 2 —¹H NMR analysis of the isomer mixture obtained in Example 1 upon adjustment of pH to 4. Part 3—¹H NMR analysis of the isomer mixture obtained in Example 1 upon adjustment of pH to 1. Part 4—HPLC-MS analysis of the isomer mixture obtained in Example 1 upon adjustment of pH to 1. Part 5—HPLC-MS analysis of the isomer mixture obtained in Example 1 upon adjustment of pH to 1 and upon spiking of the mixture with the isomer contained in the second isolated peak of Example 2.

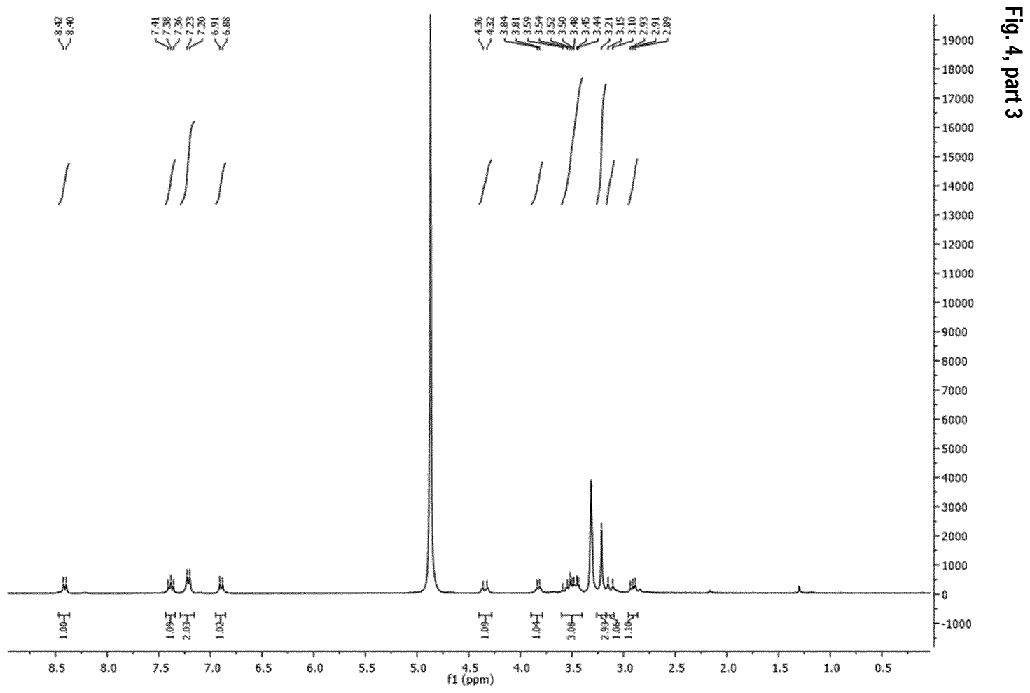

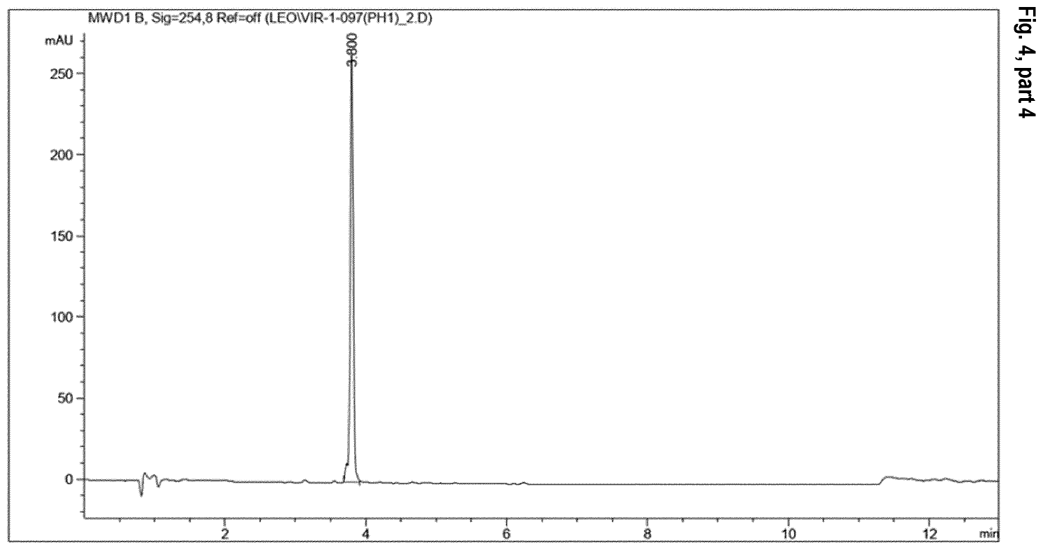

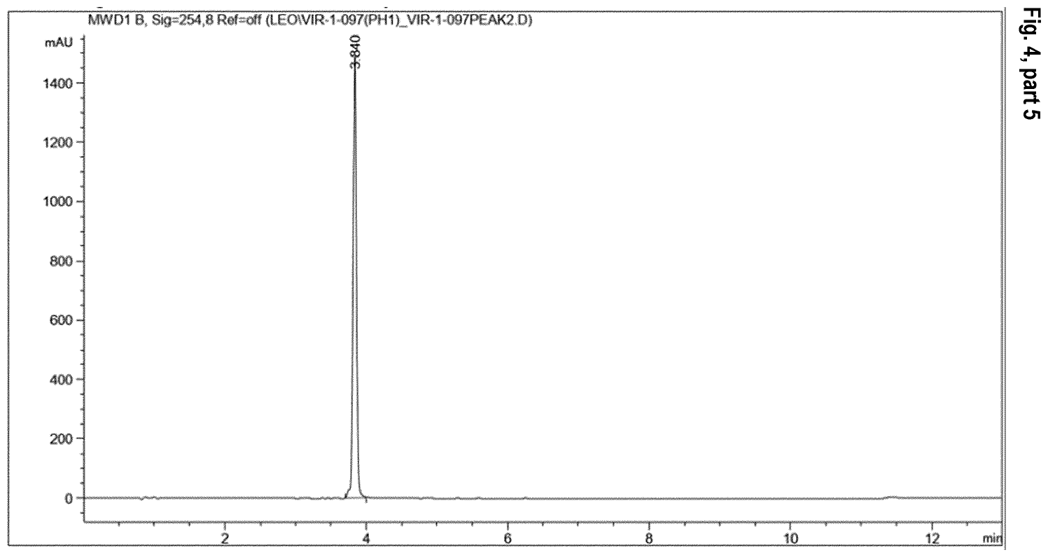

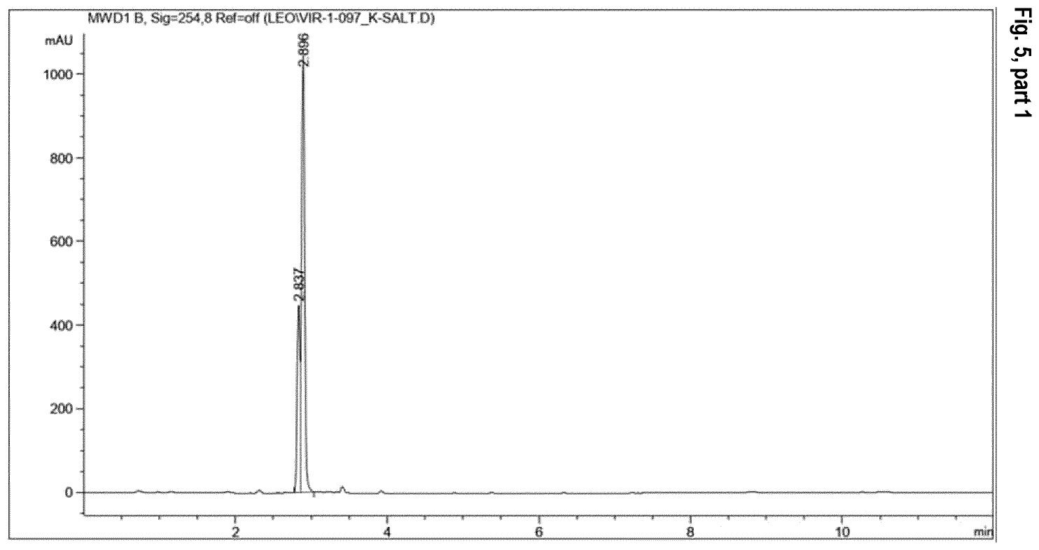

FIG. 5: HPLC-MS analysis of products of cation exchange of the apomorphine monophosphate (sodium salt) isomer mixture of Example 1 with K+, NH₄+, Ca²⁺ and Mg²⁺, as shown in Parts 1 to 4, respectively.

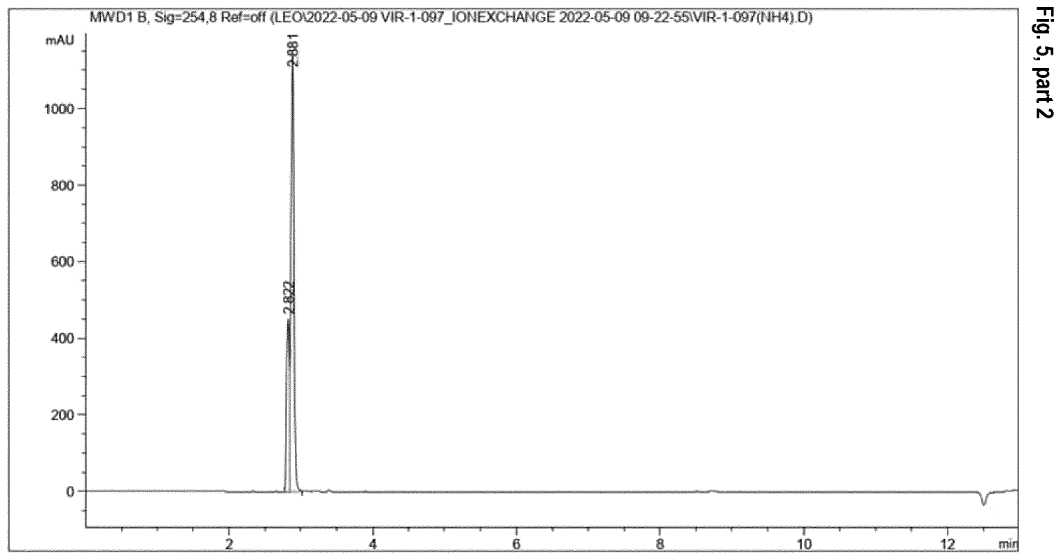

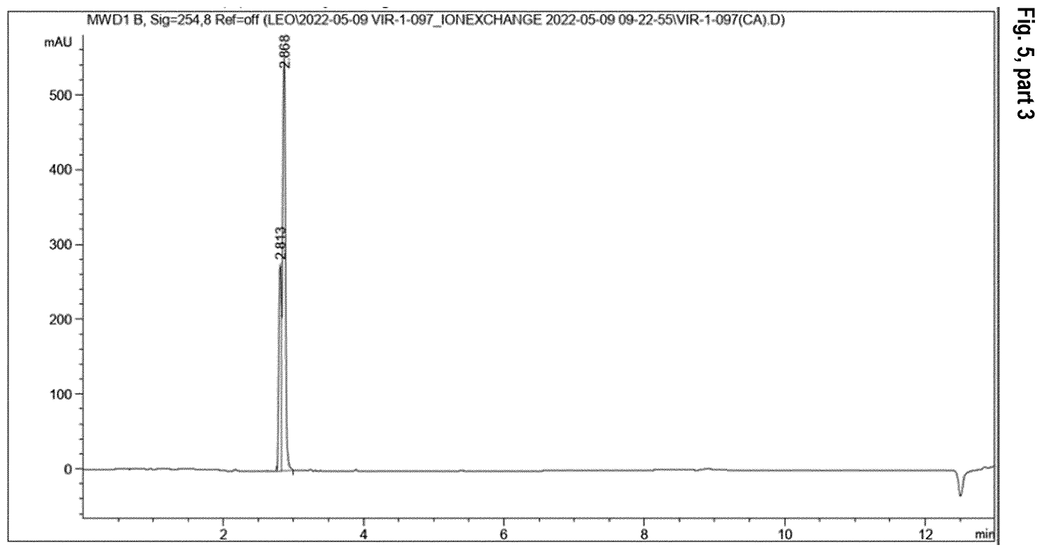

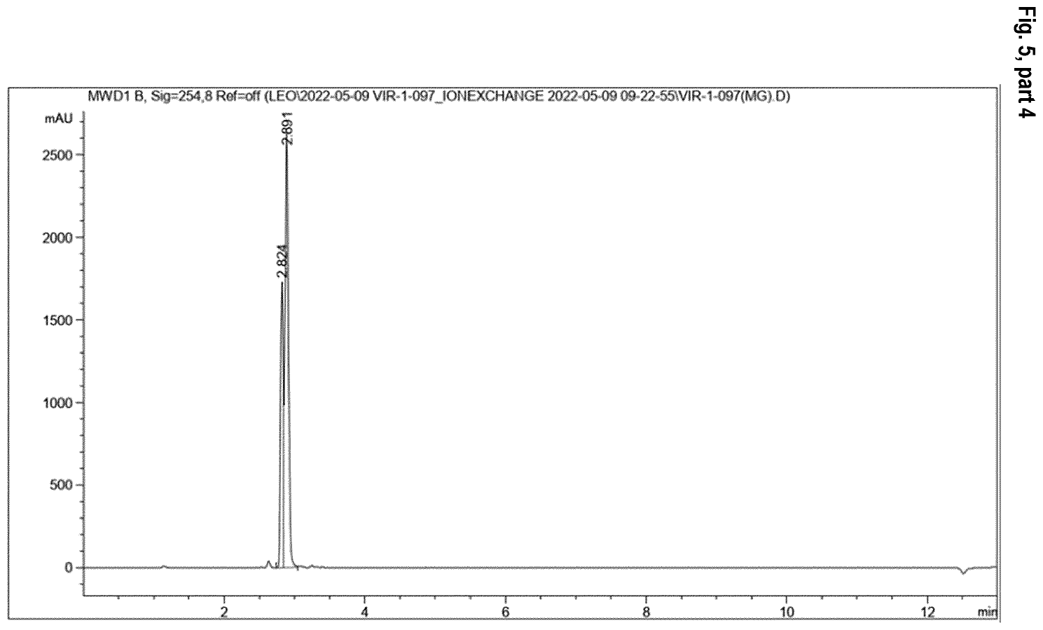

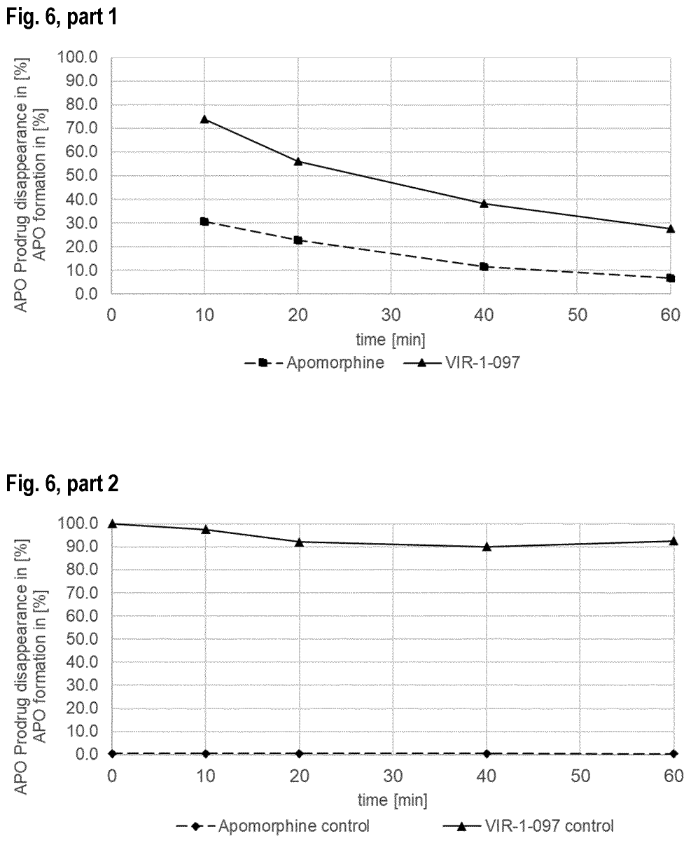

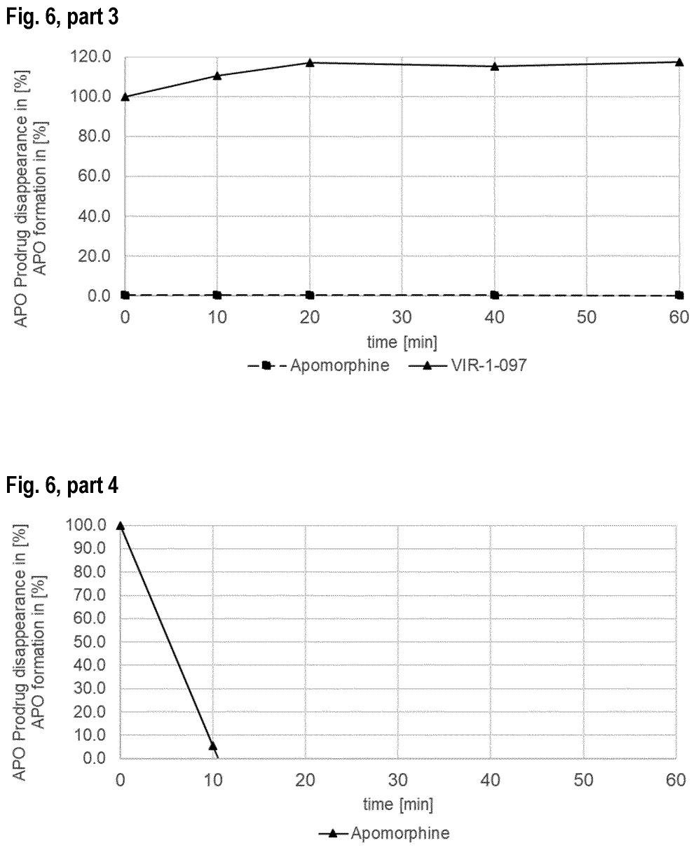

FIG. 6: Part 1—Apomorphine monophosphate in vitro stability in human hepatocytes. Part 2—Apomorphine monophosphate in vitro stability human hepatocytes control (cell-free negative control group). Part 3—Apomorphine monophosphate in vitro stability in human liver S9 homogenate. No conversion of prodrug observed. Part 4—Apomorphine HCl hemihydrate in vitro stability in human liver S9 homogenate. Apomorphine is rapidly metabolized in S9 cell extract of liver cells.

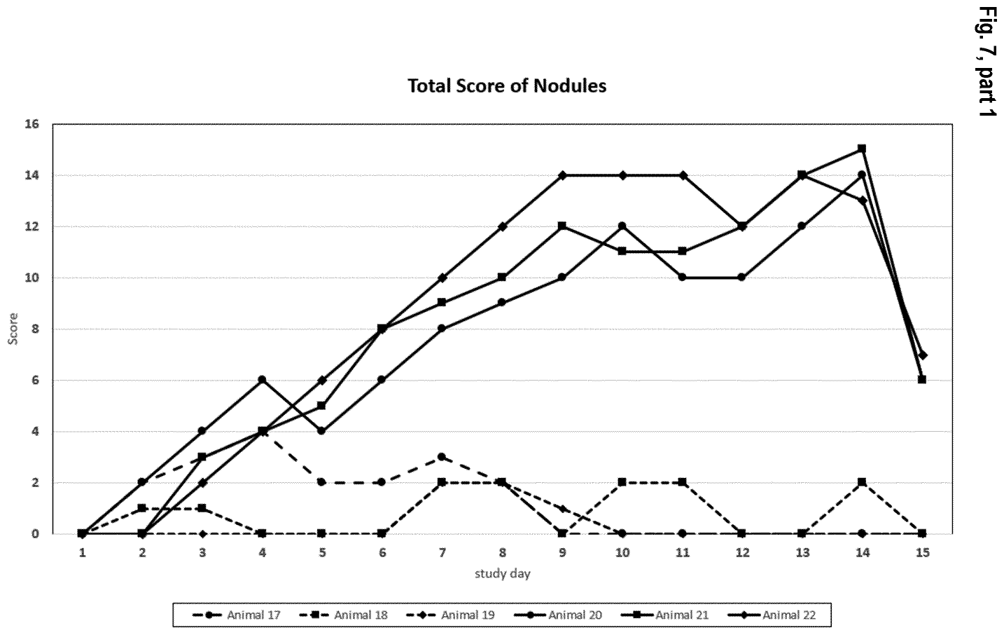

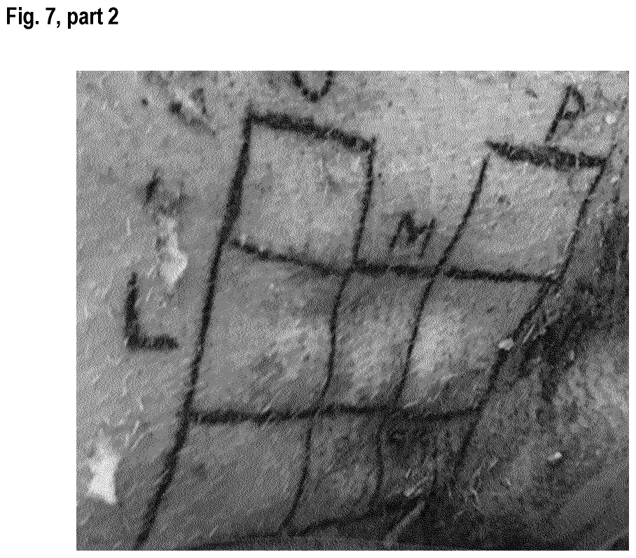

FIG. 7: Part 1—Summary of the total score for nodules as measured in the animal trial described in Example 7. Part 2—Nodules formed in the animal 22, treated with Dacepton, as seen on day 8 of the study.

The following detailed description applies to all embodiments of the present invention, including all embodiments according to each one of the first, second, third, fourth, fifth, sixth, seventh, eighth and ninth aspect as described herein above.

In the first aspect, the present invention relates to a compound of formula (I) or a pharmaceutically acceptable salt thereof:

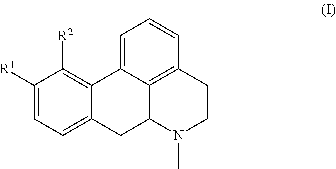

In formula (I), R¹ and R² are each independently —OH or —O—P(═O)(—OH)(—OH), provided that at least one of R¹ and R² is —O—P(═O)(—OH)(—OH).

Accordingly, R¹ may be —OH and R² may be —O—P(═O)(—OH)(—OH); or R¹ may be —O—P(═O)(—OH)(—OH) and R² may be —OH; or R¹ and R² may each be —O—P(═O)(—OH)(—OH). The compound of formula (I) may thus be, e.g., a compound having any one of the following formulae, or a pharmaceutically acceptable salt thereof:

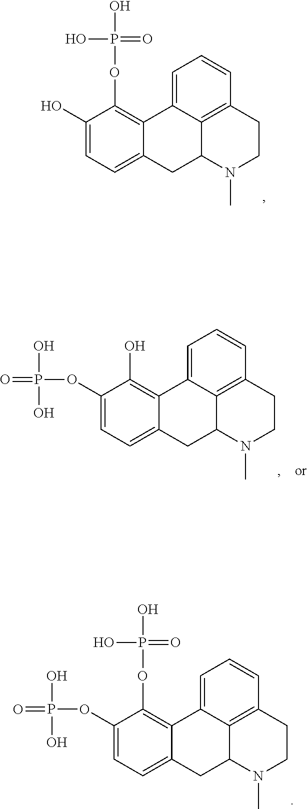 [SMILES: CN1CCc2cccc3c2C1Cc1ccc(O)c(OP(=O)(O)O)c1-3 ; CN1CCc2cccc3c2C1Cc1ccc(OP(=O)(O)O)c(O)c1-3 ; CN1CCc2cccc3c2C1Cc1ccc(OP(=O)(O)O)c(OP(=O)(O)O)c1-3]

Preferably, one of R¹ and R² is —O—P(═O)(—OH)(—OH), and the other one of R¹ and R² is —OH.

More preferably, R¹ is —OH and R² is —O—P(═O)(—OH)(—OH). Accordingly, it is particularly preferred that the compound of formula (I) is a compound having the following formula or a pharmaceutically acceptable salt thereof:

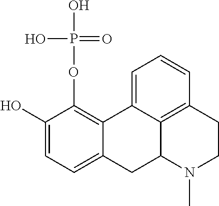 [SMILES: CN1CCc2cccc3c2C1Cc1ccc(O)c(OP(=O)(O)O)c1-3]

It is furthermore preferred that the compound of formula (I) has the (R)-configuration at the stereocenter in formula (I), i.e. at the carbon ring atom adjacent to the nitrogen ring atom. Accordingly, it is preferred that the compound of formula (I) has the following configuration:

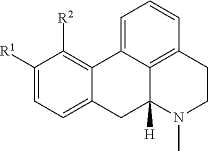

Accordingly, it is preferred that the compound of formula (I) is a compound having any one of the following formulae or a pharmaceutically acceptable salt thereof:

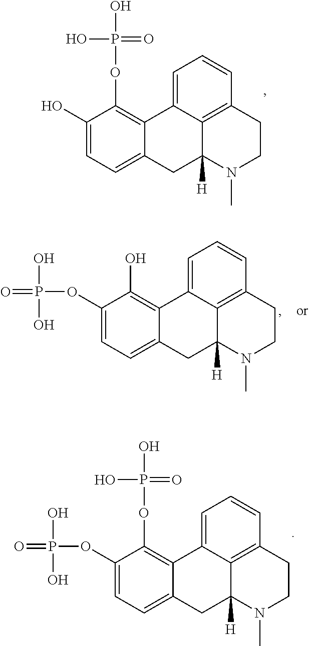

Even more preferably, the compound of formula (I) is a compound having the following formula or a pharmaceutically acceptable salt thereof:

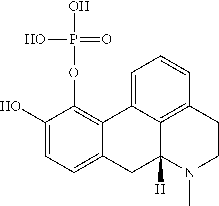 [SMILES: CN1CCc2cccc3c2[C@H]1Cc1ccc(O)c(OP(=O)(O)O)c1-3]

The compounds of formula (I) and their pharmaceutically acceptable salts can be prepared according to the methods of synthesis and/or separation described in the examples section, particularly in Examples 1 to 4. The present invention also specifically relates to each of the compounds described in the examples section.

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a phenolic —OH group or a phosphate group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), β-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred examples of a pharmaceutically acceptable salt of the compound of formula (I) include a pharmaceutically acceptable alkali metal salt or a pharmaceutically acceptable divalent metal cation salt. Particularly preferred examples of a pharmaceutically acceptable salt of the compound of formula (I) include a sodium salt, a potassium salt, an ammonium (NH₄⁺) salt, a magnesium salt, a calcium salt, a zinc salt, a copper salt (particularly a Cu(II) salt), or an iron salt (particularly an Fe(II) salt). An especially preferred example of a pharmaceutically acceptable salt of the compound of formula (I) is a sodium salt.

The present invention also specifically relates to the compound of formula (I), including any one of the specific compounds of formula (I) described herein, in non-salt form.

The compound of formula (I) or the pharmaceutically acceptable salt thereof may also be present in any solvated form, including in particular as a solvate with water. Accordingly, the compound of formula (I) or the pharmaceutically acceptable salt thereof may also be in the form of a hydrate. Moreover, the invention also encompasses all physical forms, including any amorphous or crystalline forms (i.e., polymorphs), of the compound of formula (I) or a pharmaceutically acceptable salt thereof.

For simplicity, reference will be made to the “compound of formula (I)” (or the “compounds of formula (I)”) when discussing the compound of formula (I) or a pharmaceutically acceptable salt thereof in the following. It is to be understood that any such reference relates to the compound of formula (I) or a pharmaceutically acceptable salt thereof, and also specifically relates to the corresponding compound in non-salt form or in the form of a pharmaceutically acceptable salt.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol % hydrogen 1 (¹H) and about 0.0156 mol % deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹¹C, ¹³N, and/or ¹⁵O. Such compounds can be used as tracers, trackers or imaging probes in positron emission tomography (PET). The invention thus includes (i) compounds of formula (I), in which one or more carbon atoms (or, e.g., all carbon atoms) are replaced by ¹¹C atoms, (ii) compounds of formula (I), in which the nitrogen atom is replaced by ¹³N atom, and (iii) compounds of formula (I), in which one or more oxygen atoms (or, e.g., all oxygen atoms) are replaced by ¹⁵O atoms, In general, it is preferred that none of the atoms in the compounds of formula (I) are replaced by specific isotopes.

The present invention further relates to a pharmaceutical composition comprising the compound of formula (I) and a pharmaceutically acceptable excipient. The invention particularly relates to a pharmaceutical composition comprising a compound of formula (I) or a pharmaceutically acceptable salt thereof, and a pharmaceutically acceptable excipient, for use as a medicament. The pharmaceutical composition may comprise a single compound of formula (I) or a pharmaceutically acceptable salt thereof, or it may comprise more than one compound of formula (I) or a pharmaceutically acceptable salt thereof. In particular, the invention relates to a pharmaceutical composition comprising: (i) a compound of formula (I) wherein R¹ is —OH and R² is —O—P(═O)(—OH)(—OH) (“first compound”), or a pharmaceutically acceptable salt thereof; (ii) a compound of formula (I) wherein R¹ is —O—P(═O)(—OH)(—OH) and R² is —OH (“second compound”), or a pharmaceutically acceptable salt thereof; and a pharmaceutically acceptable excipient. The molar ratio of the “first compound” to the “second compound” is not particularly limited and may be, e.g., in the range from 10:1 to 1:10, preferably in the range from 5:1 to 1:5, more preferably in the range from 3:1 to 1:3, even more preferably in the range of 2:1 to 1:2 (e.g., about 1:1).

The compounds of formula (I) provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof. A preferred antimicrobial preservative is benzyl alcohol.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, oromucosal, buccal, sublingual, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets, effervescent tablets, and sublingual films. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The pharmaceutical composition preferably comprises the compound of formula (I) or a pharmaceutically acceptable salt thereof (preferably a sodium salt) in an amount of about 0.1 mg/ml to 50 mg/ml, more preferably in an amount of about 3 mg/ml to about 20 mg/ml, and even more preferably in an amount of about 5 mg/ml to about 10 mg/ml. It is to be understood that the aforementioned amounts/concentrations are based on the weight of the compound of formula (I) or the pharmaceutically acceptable salt thereof, i.e., they are not based on the weight of the non-salt form of the compound of formula (I), unless the compound of formula (I) is actually used in the non-salt form. For example, if the compound of formula (I) is present in the form of trisodium salt, anhydrous, then 5 mg/ml refers to an amount of 5 mg of trisodium salt of the compound of formula (I) in 1 ml of the pharmaceutical composition. It is further to be understood that, accordingly, the aforementioned amounts/concentrations are not based on corresponding amounts of apomorphine or its salt that would be obtained upon hydrolysis of the compound of formula (I) or its pharmaceutically acceptable salt. The provision of the compound of formula (I) in high concentrations in a pharmaceutical composition can be facilitated, e.g., by including a solubility-enhancing agent, such as α-propylene glycol, in the pharmaceutical composition.

A pharmaceutical composition comprising the compound of formula (I) or a pharmaceutically acceptable salt thereof can be directly administered via the subcutaneous route, e.g., by subcutaneous injection. Aqueous compositions comprising higher concentrations of the compound of formula (I) or a pharmaceutically acceptable salt thereof can be used, e.g., for subcutaneous continuous infusion, where the aqueous composition may be diluted to a desired final concentration upon administration, or for subcutaneous administration by intermittent bolus injection using an injection pen, where the aqueous composition is prefilled into a pen cartridge (also referred to as a “karpule”). Accordingly, the pharmaceutical composition of the invention may be provided, e.g., in a container (such as an injection vial) or in a pen cartridge.

The pharmaceutical composition according to the invention may also comprise one or more antioxidants, such as, e.g., reduced glutathione (“GSH”; IUPAC name: (2S)-2-amino-4-{[(1R)-1-[(carboxymethyl)carbamoyl]-2-sulfanylethyl]carbamoyl}butanoic acid) or a pharmaceutically acceptable salt thereof (e.g., an alkali metal salt, particularly a sodium salt), and/or ascorbic acid (particularly L-ascorbic acid, i.e. (5R)—[(1S)-1,2-dihydroxyethyl]-3,4-dihydroxyfuran-2(5H)-one) or a pharmaceutically acceptable salt thereof (e.g., sodium ascorbate, potassium ascorbate, or calcium ascorbate). It is preferred that the pharmaceutical composition comprises both reduced glutathione (or a pharmaceutically acceptable salt thereof) and ascorbic acid (or a pharmaceutically acceptable salt thereof). Reduced glutathione (GSH) or a pharmaceutically acceptable salt thereof may be present in the pharmaceutical composition of the invention in an amount of about 1 mg/ml to about 50 mg/ml, more preferably in an amount of about 1 mg/ml to about 20 mg/ml, even more preferably in an amount of about 2 mg/ml to about 10 mg/ml, and still more preferably in an amount of about 5 mg/ml. The pharmaceutical composition of the invention may comprise may be present in the pharmaceutical composition of the invention in an amount of about 2 mg/ml to about 50 mg/ml, more preferably in an amount of about 5 mg/ml to about 20 mg/ml, even more preferably in an amount of about 8 mg/ml to about 15 mg/ml, and yet even more preferably in an amount of about 10 mg/ml.

The pharmaceutical composition of the invention is preferably an aqueous pharmaceutical composition. Accordingly, it is preferred that the pharmaceutical composition comprises water, particularly at least about 60% (v/v) water, more preferably at least about 70% (v/v) water, even more preferably at least about 80% (v/v) water, even more preferably at least about 90% (v/v) water, and yet even more preferably at least about 95% (v/v) water, with respect to the total volume of the pharmaceutical composition. The water in the pharmaceutical composition is preferably water for injection (e.g., as defined in the European Pharmacopoeia (Ph. Eur.), 8th Edition as of Jul. 1, 2015, including supplement 8.6). Water for injection (WFI) can be prepared using techniques known in the art, e.g., by distillation or by membrane technologies (such as reverse osmosis or ultrafiltration), as described, e.g., in Felton L A (ed.), Remington: Essentials of Pharmaceutics, Pharmaceutical Press, 2013.

The pharmaceutical composition may be, e.g., an aqueous solution or an oil-in-water emulsion. In this regard, it is preferred that the pharmaceutical composition has an oil content of less than about 5% (v/v), more preferably of less than about 3% (v/v), even more preferably of less than about 2% (v/v), even more preferably of less than about 1% (v/v), even more preferably of less than about 0.5% (v/v), and yet even more preferably it does not contain any oil. Accordingly, it is preferred that the pharmaceutical composition is an aqueous solution.

Furthermore, it is preferred that the pharmaceutical composition has a total content of lipophilic substances of less than about 5% (v/v), more preferably of less than about 3% (v/v), even more preferably of less than about 2% (v/v), even more preferably of less than about 1% (v/v), even more preferably of less than about 0.5% (v/v), and yet even more preferably it does not contain any lipophilic substances.

The pharmaceutical composition is preferably an aqueous pharmaceutical composition having a pH of about 3 to about 7.4 (such as, e.g., a pH of about 3.0, about 3.7, about 4.0, about 4.5, about 5.0, about 5.5, about 6.0, about 6.5, about 7.0, or about 7.4). More preferably, it has a pH of about 4 to about 7, more preferably a pH of about 5 to about 6 (e.g., about 5.0, about 5.5, or about 6.0), and even more preferably a pH of about 5.5.

The desired pH may be controlled by using a suitable buffer and, if necessary, adjusting the pH by adding an aqueous solution of HCl or of NaOH. The buffer is not particularly limited and may be selected, e.g., from malate buffer, formate buffer, succinate buffer, citrate buffer, acetate buffer, pyridine buffer, MES buffer, tartrate buffer, oxalate buffer, ascorbate buffer, cacodylate buffer, dimethylglutarate buffer, carbonate buffer, Bis-Tris buffer, ADA buffer, pyrophosphate buffer, EDPS buffer, Bis-Tris propane buffer, PIPES buffer, ACES buffer, MOPSO buffer, imidazole buffer, histidine buffer, BES buffer, MOPS buffer, phosphate buffer, EMTA buffer, TES buffer, HEPES buffer, and DIPSO buffer (as described, e.g., in Stoll V S, et al. Buffers: principles and practice. Methods Enzymol. 1990. 182:24-38; or in AppliChem, Biological Buffers, 2008). It will be understood that a suitable buffer can be selected depending on the pKₐ value of the buffer substance and the desired pH of the pharmaceutical composition.

The pharmaceutical composition according to the invention may further comprise α-propylene glycol (i.e., propane-1,2-diol) and/or sodium chloride. These substances can be used as isotonizing agents for rendering the pharmaceutical composition isotonic with human blood plasma. Accordingly, the pharmaceutical composition may comprise α-propylene glycol in an amount of, e.g., about 1 mg/ml to about 25 mg/ml (or about 5 mg/ml to about 15 mg/ml), but if α-propylene glycol is used as an isotonizing agent, the preferred amount of α-propylene glycol will depend on the pH of the pharmaceutical composition (e.g., a pharmaceutical composition according to the invention having a pH of about 5 may preferably contain α-propylene glycol in an amount of about 5 mg/ml to about 10 mg/ml).

The pharmaceutical composition may comprise sodium chloride in an amount of, e.g., about 3 mg/ml to about 8 mg/ml, but if sodium chloride is used as an isotonizing agent, the preferred amount of sodium chloride will depend on the pH of the pharmaceutical composition (e.g., a pharmaceutical composition according to the invention having a pH of about 5 may preferably contain sodium chloride in an amount of about 4.5 mg/ml).

It is preferred that the pharmaceutical composition is isotonic with respect to human blood plasma. In particular, it is preferred that the pharmaceutical composition has an osmolality of about 280 mOsm/kg to about 305 mOsm/kg, more preferably an osmolality of about 290 mOsm/kg to about 300 mOsm/kg, and even more preferably an osmolality of about 296 mOsm/kg. Moreover, the pharmaceutical composition is preferably rendered isotonic (e.g., to any of the aforementioned osmolality ranges or values) using α-propylene glycol and/or sodium chloride, more preferably using α-propylene glycol, as described hereinabove.

In principle, the compounds of formula (I) or the pharmaceutically acceptable salts thereof or the above-described pharmaceutical compositions may be administered to a subject by any convenient route of administration. Corresponding routes of administration include but are not limited to: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration. The compound of formula (I) or the pharmaceutical composition is preferably administered parenterally (e.g., by injection or infusion), more preferably subcutaneously.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. Thus, for example, parenteral administration of the compounds of the present invention may be administration through subcutaneous injection or infusion. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions may also be administered orally in the form of tablets, capsules, ovules, elixirs, solutions or suspensions, which may contain flavoring or coloring agents, for immediate-, delayed-, modified-, sustained-, pulsed- or controlled-release applications. The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof. For oral administration, the compounds or the pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions may be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi permeable polymer matrices in the form of shaped articles, e.g., films, or microcapsules. Sustained-release matrices include, e.g., polylactides, copolymers of L glutamic acid and gamma-ethyl-L-glutamate, poly(2-hydroxyethyl methacrylate), ethylene vinyl acetate, or poly D (−)-3-hydroxybutyric acid. Sustained-release pharmaceutical compositions also include liposomally entrapped compounds. The present invention thus also relates to liposomes containing a compound of the invention or a pharmaceutically acceptable salt thereof.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) or the pharmaceutically acceptable salt thereof for pulmonary administration, particularly inhalation. Such dry powders may be prepared by spray drying under conditions which result in a substantially amorphous glassy or a substantially crystalline bioactive powder. Accordingly, dry powders of the compounds of the present invention may be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compound of formula (I) or a pharmaceutically acceptable salt thereof, or a pharmaceutical composition as described herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. A preferred route of administration is parenteral administration, in particular subcutaneous administration (e.g., through subcutaneous injection or infusion) or intramuscular administration. A particularly preferred route of administration is subcutaneous administration, e.g., through subcutaneous injection or infusion. Accordingly, for each of the compounds or pharmaceutical compositions provided herein, it is particularly preferred that the respective compound or pharmaceutical composition is to be administered subcutaneously (particularly by subcutaneous injection or infusion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

For example, the compound or the pharmaceutical composition according to the invention can be administered to a human subject (preferably a human subject aged 18 or older) by subcutaneous intermittent bolus injection in a dose of about 1 mg to about 10 mg of the active ingredient (i.e., the compound of formula (I) or the pharmaceutically acceptable salt thereof). The corresponding unit dose may, e.g., be administered subcutaneously 1-12 times per day (preferably 1-10 times per day), up to a maximum daily dose of, e.g., about 100 mg (preferably up to a maximum daily dose of about 50 mg, and more preferably up to a maximum daily dose of about 30 mg). Alternatively, the compound or the pharmaceutical composition can also be administered to a human subject (preferably a human subject aged 18 or older) by subcutaneous continuous infusion (e.g., using a minipump and/or a syringe driver) at an infusion rate of about 1 mg to about 4 mg of the active ingredient (i.e., the compound of formula (I) or the pharmaceutically acceptable salt thereof, which may be comprised in a pharmaceutical composition in a concentration of, e.g., about 5 mg/ml) per hour, or at an infusion rate of about 0.015 mg/kg/hour to about 0.06 mg/kg/hour of the active ingredient (i.e., the compound of formula (I) or the pharmaceutically acceptable salt thereof, which may be comprised in a pharmaceutical composition in a concentration of, e.g., about 5 mg/ml). It will be appreciated that it may be necessary to make routine variations to the dosage depending on the age and weight of the patient/subject as well as the severity of the condition to be treated. The precise dose and also the route of administration will ultimately be at the discretion of the attendant physician (or attendant veterinarian).

Apomorphine has been described to be useful in the treatment of Parkinson's disease (e.g., Schwab R S, et al. Trans Am Neurol Assoc. 1951. 56:251-253; Cotzias G C, et al. N Engl J Med. 1970. 282:31-33; Poewe W, et al. Mov Disord. 2000. 15(5):789-794; and Manson A J, et al. Mov Disord. 2002. 17(6):1235-1241), Alzheimer's disease (e.g., Lashuel H A, et al. J Biol Chem. 2002. 277(45):42881-42890; Steele J W, et al. Ann Neurol. 2011. 69(2):221-225; and Himeno E, et al. Ann Neurol. 2011. 69(2):248-256), Huntington's disease (e.g., Corsini G U, et al. Arch Neurol. 1978. 35(1):27-30; Colosimo C, et al. Clin Neuropharmacol. 1994. 17(3):243-259; and Albanese A, et al. Clin Neuropharmacol. 1995. 18(5):427-434), neuroleptic malignant syndrome (e.g., Colosimo C, et al. Clin Neuropharmacol. 1994. 17(3):243-259; and Wang H C, et al. Mov Disord. 2001. 16(4):765-767), dystonia (e.g., Colosimo C, et al. Clin Neuropharmacol. 1994. 17(3):243-259), schizophrenia (e.g., Smith R C, et al. J Neural Transm. 1977. 40(2):171-176; and Tamminga C A, et al. Science. 1978. 200(4341):567-568), and further neurodegenerative diseases/disorders (e.g., Kyriazis M. J Anti Aging Med. 2003. 6(1):21-28; and Truong J G, et al. Eur J Pharmacol. 2004. 492(2-3):143-147). The use of apomorphine for the treatment of erectile dysfunction and impotence has also been described in the literature (e.g., Heaton J P, et al. Urology. 1995. 45(2):200-206; O'Sullivan J D, et al. Mov Disord. 1998. 13(3):536-539; Dula E, et al. Urology. 2000. 56(1):130-135; and Rampin 0. BJU nt. 2001. 88 Suppl. 3:22-24). As the compound of formula (I) can be used as a prodrug of apomorphine, it can be used for the treatment of any the above-mentioned disorders as well as any other disorders for which the use of apomorphine has been proposed in the literature.

As used herein, the term “treatment” (or “treating”) in relation to a disease or disorder refers to the management and care of a patient for the purpose of combating the disease or disorder, such as to reverse, alleviate, inhibit or delay the disease or disorder, or one or more symptoms of such disease or disorder. It also refers to the administration of a compound or a composition for the purpose of preventing the onset of symptoms of the disease or disorder, alleviating such symptoms, or eliminating the disease or disorder. The “treatment” may be prophylactic or non-prophylactic. Preferably, the “treatment” is curative, ameliorating or palliative.

The pharmaceutical composition according to the invention, comprising the compound of formula (I) or a pharmaceutically acceptable salt thereof as active agent, may be administered in the context of a monotherapy or in combination with one or more further pharmaceutically active agents. If the pharmaceutical composition of the invention is used in combination with a further pharmaceutically active agent which is active against the same disease/disorder, a lower dose of each agent may be used. The combination of the pharmaceutical composition according to the present invention with one or more further pharmaceutically active agents may comprise the simultaneous/concomitant administration of the further pharmaceutically active agents with the pharmaceutical composition according to the invention. However, sequential/separate administration is also envisaged. When administration is sequential, either the pharmaceutical composition of the invention or the one or more further pharmaceutically active agents may be administered first. When administration is simultaneous, the one or more further pharmaceutically active agents may be included in the pharmaceutical composition of the invention or may be administered in one or more different (separate) pharmaceutical compositions.

For the treatment of Parkinson's disease, the compound of formula (I) (or a pharmaceutically acceptable salt thereof) or the pharmaceutical composition according to the invention can be administered in combination with one or more further antiparkinson agents which may, for example, be selected from etilevodopa, droxidopa, levodopa, melevodopa, aplindore, bromocriptine, cabergoline, ciladopa, dihydroergocryptine, lisuride, pardoprunox, pergolide, piribedil, pramipexole, ropinirole, rotigotine, ladostigil, lazabemide, mofegiline, pargyline, rasagiline, selegiline, entacapone, nitecapone, tolcapone, benserazide, carbidopa, methyldopa, benzatropine, biperiden, bornaprine, chlorphenoxamine, cycrimine, dexetimide, dimenhydrinate, diphenhydramine, etanautine, etybenzatropine, mazaticol, metixene, orphenadrine, phenglutarimide, piroheptine, procyclidine, profenamine, trihexyphenidyl, tropatepine, amantadine, budipine, memantine, methylxanthines, rimantadine, UWA-101, and pharmaceutically acceptable salts of any of these agents. Preferred antiparkinson agents are levodopa, carbidopa, and biperiden. A particularly preferred antiparkinson agent is levodopa.

Accordingly, the present invention relates to the compound of the first aspect, or the pharmaceutical composition of the second aspect, as described and defined herein, for use in the treatment of Parkinson's disease (or for use in the treatment of refractory motor fluctuations/oscillations in Parkinson's disease, off-periods in Parkinson's disease, refractory off-periods in Parkinson's disease, dyskinesia in Parkinson's disease, and/or akinesia in Parkinson's disease), wherein said compound or said pharmaceutical composition is to be administered subcutaneously, and wherein said compound or said pharmaceutical composition is to be administered in combination with one or more further antiparkinson agents (e.g., one or more of the specific antiparkinson agents described above). The combined administration of the compound or the pharmaceutical composition with one or more further antiparkinson agents may be effected, e.g., by simultaneous/concomitant administration or by sequential/separate administration. The one or more further antiparkinson agents are not necessarily administered subcutaneously but may rather be administered by any convenient route of administration.

Since apomorphine, the product of hydrolysis of the compounds of formula (I), can cause short-term nausea, particularly in the beginning of the treatment, the compound of the invention or the pharmaceutical composition according to the invention is preferably administered in combination with an anti-emetic agent. Such a combination treatment may, for example, be administered for a period of at least two weeks before the administration of the anti-emetic agent may be terminated while the administration of the compound or of the pharmaceutical composition of the invention is continued. The anti-emetic agent may, for example, be selected from alizapride, alosetron, aprepitant, atropine, azasetron, bemesetron, benzquinamine, bromopride, buclizine, casopitant, cerium oxalate, chlorpromazine, cilansetron, clebopride, clozapine, cyclizine, cyproheptadine, dazopride, dexamethasone, dimenhydrinate, diphenhydramine, diphenidol, dolasetron, domperidone, dronabinol, ezlopitant, fosaprepitant, granisetron, haloperidol, hydroxyzine, hyoscyamine, itopride, lerisetron, lorazepam, maropitant, meclozine, metoclopramide, metopimazine, mianserin, midazolam, mirtazapine, nabilone, nonabine, olanzapine, ondansetron, oxypendyl, palonosetron, pipamazine, prochlorperazine, promethazine, propofol, quetiapine, ramosetron, ricasetron, risperidone, scopolamine, tetrahydrocannabinol, thiethylperazine, trimethobenzamide, tropisetron, vestipitant, zatosetron, ziprasidone, and pharmaceutically acceptable salts (e.g., a hydrochloride) of any of these agents. A particularly preferred anti-emetic agent is domperidone.

Accordingly, the present invention relates to a compound of formula (I) or a pharmaceutical composition comprising the same, as described and defined herein, wherein said compound or said pharmaceutical composition is to be administered subcutaneously, and wherein said compound or said pharmaceutical composition is to be administered in combination with an anti-emetic agent (e.g., any one of the anti-emetic agents listed above, preferably domperidone). The combined administration of the compound or the pharmaceutical composition with an anti-emetic agent may be effected, e.g., by simultaneous/concomitant administration of the anti-emetic agent with the compound or the pharmaceutical composition or, alternatively, by sequential/separate administration. When administration is sequential, either the compound/pharmaceutical composition of the invention or the anti-emetic agent may be administered first. When administration is simultaneous, the anti-emetic agent may be included in the pharmaceutical composition or may be administered in a different (separate) pharmaceutical composition. The anti-emetic agent, if provided in a separate pharmaceutical composition, does not need to be administered subcutaneously but may rather be administered by any convenient route of administration.

The invention also relates to the combined administration of the compound or the pharmaceutical composition of the invention with an anti-emetic agent (as described above) and with one or more further antiparkinson agents (as described above).

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

As used herein, the term “about” preferably refers to ±10% of the indicated numerical value, more preferably to ±5% of the indicated numerical value, and in particular to the exact numerical value indicated.

The terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

The term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

Any parameters referred to herein (including, e.g., any amounts/concentrations indicated in “mg/ml” or in “% (v/v)”, and any pH values) are preferably to be determined at standard ambient temperature and pressure conditions, particularly at a temperature of 25° C. (298.15 K) and at an absolute pressure of 1 atm (101.325 kPa).

It is to be understood that the present invention specifically relates to each and every combination of features and embodiments described herein, including any combination of general and/or preferred features/embodiments.

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLES

### Example 1: Synthesis of Apomorphine Monophosphate

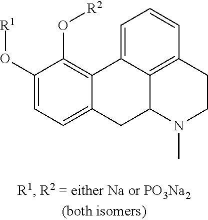

In a flame-dried and argon-purged Schlenk flask, a suspension of apomorphine hydrochloride hemihydrate (10.0 g, 32.0 mmol) in anhydrous CH₂Cl₂ (80 mL) was cooled to 0° C. via an ice/water-bath and deprotonated using Et₃N (13.5 mL, 9.86 g, 97.4 mmol). After stirring for 20 min at 0° C., phosphoryl chloride (6.40 mL, 10.8 g, 70.1 mmol) was added and the resulting yellowish suspension was heated under reflux for 66 h. After cooling to room temperature (rt), the solvent was removed under reduced pressure (rotary evaporator) and the residue was taken up in water (100 mL). The pH was adjusted to pH ˜11 by addition of 3 M aq. NaOH. The resulting dark-green suspension was filtered through a sintered glass frit and the filtrate was directly loaded on a reversed-phase silica gel column (50 mL C18 silica gel, 400-200 mesh; eluent: H₂O; fraction size: 15 mL; fraction analysis: HPLC). Fractions containing the desired product were pooled and the pH was adjusted to pH ˜5 by addition of 1 M HCl, resulting in an off-white suspension. The formed precipitate (protonated apomorphine monophosphate) was collected by filtration, suspended in 100 mL H₂O and deprotonated with 3 M NaOH (pH˜10). The resulting dark-green solution was lyophilized to give the desired product.
Yield 4.60 g (11.1 mmol, 35%), beige powder, C₁₇H1₅NNa₃O5P [413.25]
M.p.>210° C. decomposition (Na-salt)
HRMS calcd (M/z) for [C₁₇H1₈NO₅P-H+]: 348.0995; found: 348.1000.

NMR spectra of the obtained product are shown in FIG. 1.

¹H NMR (300.36 MHz, D₂O) δ=8.38 (d, J=7.6 Hz, 1H, both isomers), 7.37 (s, 1H, isomer a), 7.29 (t, J=7.7 Hz, 1H, isomer b), 7.23-7.07 (m, 2H, both isomers), 7.00 (s, 1H, isomer a), 6.86 (d, J=8.1 Hz, 1H, isomer a), 6.56 (d, J=7.9 Hz, 1H, isomer b), 3.25-3.03 (m, 4H, both isomers), 2.80 (d, J=16.4 Hz, 1H, both isomers), 2.66-2.55 (m, 1H, both isomers), 2.50 (s, 3H, both isomers), 2.37 (m, 1H, both isomers) ppm.

¹³C NMR (75.53 MHz, D₂O) δ=148.3, 143.9, 134.5, 133.7, 133.4, 132.7, 132.6, 131.6, 131.0, 129.0, 128.0, 126.8, 126.7, 126.4, 125.6, 123.5, 122.2, 119.0, 117.1, 114.5, 61.9, 61.7, 52.0 (both isomers), 42.6, 42.5, 33.9, 33.4, 28.0 (both isomers) ppm.

HPLC-MS analysis:
Column: C-18-Reversed-Phase column of the type “Poroshell® 120 SB—C18, 3.0×100 mm, 2.7 μm” by Agilent Technologies. Flow: Constant flow rate 0.7 mL/min, T=35° C. HPLC-MS analysis is shown in FIG. 2.
Method: 0.0-0.1 min, isocratic, 2% MeCN (98% H2O+0.05% TFA); 0.1-8.0 min, linear, 2% to 100% MeCN (98% to 0% H2O+0.05% TFA); 8.0-11.1 min, isocratic, 100% MeCN; 11.1-11.3 min, linear, 100% to 2% MeCN (0% to 98% H2O+0.05% TFA); 11.3-12.0 min, isocratic, 2% MeCN (98% H2O+0.05% TFA). UV detection at 254.8 nm.

### Example 2: Separation of Regioisomers of Apomorphine Monophosphate

Purification was performed on a “Thermo Scientific Dionex UltiMate 3000” system with UltiMate 3000 pump, UltiMate 3000 autosampler, UltiMate 3000 column compartment, UltiMate 3000 diode array detector (deuterium lamp, A=190-380 nm) and an UltiMate 3000 automatic fraction collector. The components were separated on a Macherey-Nagel V P 125/21 Nucleodur® 100-5 C18ec column (125×21 mm, 5 μm). Signals were detected at 210 nm and 254 nm. As mobile phase acetonitrile (VWR HiPerSolv, HPLC grade) and water (Barnstead NANOpure*, ultrapure water system) with trifluoroacetic acid were used. The following method was used:

NucleodurC18 005TFA 02to50: 0.0-3.0 min: 98% H₂O (0.05% TFA) and 2% CH₃CN; 3.0-13.0 min: linear gradient to 50% H₂O (0.05% TFA) and 50% CH₃CN; 13.0-14.0 min: linear gradient to 100% CH₃CN; 14.0-15.0 min: 100% CH₃CN; 15.0-16.0 min: linear gradient to 98% H₂O (0.05% TFA) and 2% CH₃CN; 16.0-19.0 min: 98% H₂O (0.05% TFA) and 2% CH₃CN; flow rate: 15.00 mL·min⁻¹; T=30° C.

HPLC-MS analysis of mixture:
Column: C-18-Reversed-Phase column of the type “Poroshell® 120 SB—C18, 3.0×100 mm, 2.7 μm” by Agilent Technologies. Flow: Constant flow rate 0.7 mL/min, T=35° C.
Method: 0.0-0.1 min, isocratic, 2% MeCN (98% H2O+0.05% TFA); 0.1-10.0 min, linear gradient, 2% to 50% MeCN (98% to 0% H2O+0.05% TFA); 10.0-10.5 min, linear gradient, 50% to 100% MeCN (0% to 98% H2O+0.05% TFA); 10.5-12.0 min, isocratic, 100% MeCN; 12.0-12.5 min, linear gradient, 100% to 2% MeCN (0% to 98% H2O+0.05% TFA); isocratic,12.5-14.0 min, isocratic 2% MeCN (98% H2O+0.05% TFA). UV detection at 254.8 nm.

The results of the analysis are shown in FIG. 3, part 1.

HPLC-MS analysis of separated peak 1:
Column: C-18-Reversed-Phase column of the type “Poroshell® 120 SB—C18, 3.0×100 mm, 2.7 μm” by Agilent Technologies. Flow: Constant flow rate 0.7 mL/min, T=35° C.
Method: 0.0-0.1 min, isocratic, 2% MeCN (98% H₂O+0.05% TFA); 0.1-10.0 min, linear gradient, 2% to 50% MeCN (98% to 0% H₂O+0.05% TFA); 10.0-10.5 min, linear gradient, 50% to 100% MeCN (0% to 98% H₂O+0.05% TFA); 10.5-12.0 min, isocratic, 100% MeCN; 12.0-12.5 min, linear gradient, 100% to 2% MeCN (0% to 98% H₂O+0.05% TFA); isocratic, 12.5-14.0 min, isocratic 2% MeCN (98% H₂O+0.05% TFA). UV detection at 254.8 nm.

The results of the analysis are shown in FIG. 3, part 2.

### NMR Analysis of the Separated Peak 1

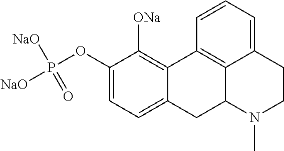

¹H NMR (300.36 MHz, D₂O) δ=8.29 (d, J=7.6 Hz, 1H), 7.19 (t, J=7.3 Hz, 1H), 6.98 (d, J=7.5 Hz, 1H), 6.71 (d, J=7.7 Hz, 2H), 3.73 (d, J=12.5 Hz, 1H), 3.55 (d, J=7.1 Hz, 1H), 3.39-3.23 (m, 1H), 3.07-2.72 (m, 6H), 2.41 (t, J=13.6 Hz, 1H) ppm.

¹³C NMR (75.53 MHz, D₂O) δ=148.5, 138.8, 131.2, 129.2, 127.9, 127.9, 127.8, 127.3, 125.9, 125.5, 123.8, 117.3, 61.2, 51.5, 30.9, 25.2 ppm.

³¹P NMR (202.35 MHz, D₂O) δ=2.47 ppm.

The ¹H, ¹³C and ³¹P NMR spectra are shown in FIG. 3 parts 3 to 5, respectively.

HPLC-MS analysis of separated peak 2:
Column: C-18-Reversed-Phase column of the type, Poroshell@120 SB—C18, 3.0×100 mm, 2.7 μm” by Agilent Technologies. Flow: Constant flow rate 0.7 mL/min, T=35° C.
Method: 0.0-0.1 min, isocratic, 2% MeCN (98% H2O+0.05% TFA); 0.1-10.0 min, linear gradient, 2% to 50% MeCN (98% to 0% H2O+0.05% TFA); 10.0-10.5 min, linear gradient, 50% to 100% MeCN (0% to 98% H2O+0.05% TFA); 10.5-12.0 min, isocratic, 100% MeCN; 12.0-12.5 min, linear gradient, 100% to 2% MeCN (0% to 98% H2O+0.05% TFA); isocratic, 12.5-14.0 min, isocratic 2% MeCN (98% H2O+0.05% TFA). UV detection at 254.8 nm.

The results of the analysis are shown in FIG. 3, part 6.

### NMR Analysis of the Separated Peak 2

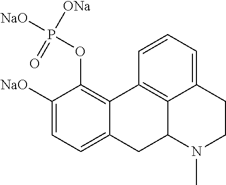

¹H NMR (300.36 MHz, D₂O) δ=8.19 (d, J=7.7 Hz, 1H), 7.30 (t, J=7.7 Hz, 1H), 7.12 (d, J=7.4 Hz, 1H), 7.01 (d, J=7.9 Hz, 1H), 6.70 (d, J=8.0 Hz, 1H), 3.45-3.28 (m, 2H), 3.23-3.04 (m, 2H), 2.84 (d, J=13.9 Hz, 2H), 2.72 (s, 3H), 2.41 (t, J=13.8 Hz, 1H) ppm.

¹³C NMR (75.53 MHz, D₂O) δ=145.7, 141.8, 141.7, 131.6, 131.1, 130.6, 130.1, 127.7, 127.4, 126.5, 121.5, 121.1, 119.7, 61.4, 51.8, 41.1, 32.0, 26.2 ppm.

³¹P NMR (202.35 MHz, D₂O) δ=2.61 ppm.

### Example 3: Ionization States of Apomorphine Monophosphate

The pKa values of apomorphine monophosphate are estimated/predicted as follows (only one isomer shown for better demonstration):

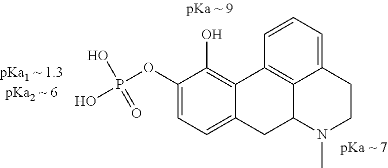 [SMILES: CN1CCc2cccc3c2C1Cc1ccc(OP(=O)(O)O)c(O)c1-3]

The following compounds were isolated upon adjusting the pH to the stated values with dropwise addition of 1 M aq. HCl to an aqueous solution of apomorphine monophosphate (trisodium salt, pH of solution ˜11, as obtained in Example 1) and lyophilization:
pH8:

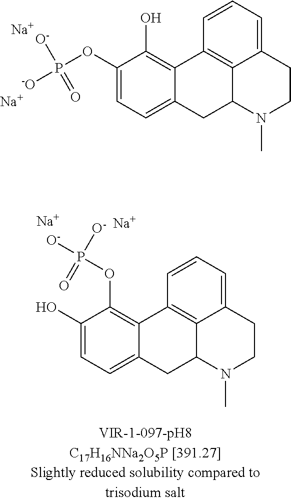

¹H NMR (300.36 MHz, DMSO-d₆) δ=8.44 (d, J=7.4 Hz, 1H, isomer 1), 8.22 (d, J=7.7 Hz, 1H, isomer 2), 7.15 (dd, J=15.2, 7.6 Hz, 1H, both isomers), 6.98 (d, J=7.1 Hz, 1H, both isomers), 6.87-6.55 (m, 2H, both isomers), 3.11-2.93 (m, 5H, both isomers), 2.69 (d, J=16.7 Hz, 1H, both isomers), 2.45 (s, 3H, both isomers), 2.37-2.19 (m, 2H, both isomers) ppm.

The corresponding ¹H NMR spectrum in DMSO-d₆ is shown in FIG. 4, part 1.
pH 4:

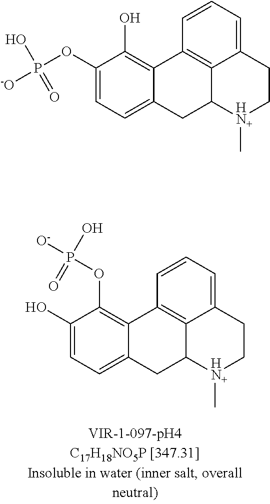

¹H NMR (300.36 MHz, DMSO-d₆) δ=8.56 (d, J=7.7 Hz, 1H, isomer 1), 8.33 (d, J=7.7 Hz, 1H, isomer 2), 7.29 (dd, J=15.5, 7.6 Hz, 1H, both isomers), 7.11 (d, J=6.9 Hz, 1H, both isomers), 6.91-6.61 (m, 2H, both isomers), 4.11 (s, 2H, both isomers), 3.43-3.23 (m, 5H, both isomers), 2.92 (s, 3H, both isomers), 2.82-2.68 (m, 1H, both isomers) ppm.

The corresponding ¹H NMR spectrum in DMSO-d₆ is shown in FIG. 4, part 2.
pH<1:

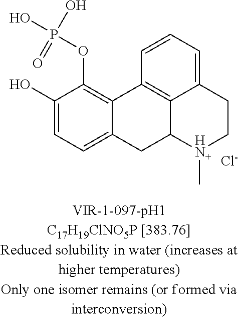

¹H NMR (300.36 MHz, CD₃OD) δ=8.41 (d, J=7.9 Hz, 1H), 7.38 (t, J=7.7 Hz, 1H), 7.21 (d, J=7.7 Hz, 2H), 6.90 (d, J=8.1 Hz, 1H), 4.34 (d, J=11.1 Hz, 1H), 3.83 (d, J=7.3 Hz, 1H), 3.61-3.42 (m, 3H), 3.21 (s, 3H), 3.13 (d, J=14.0 Hz, 1H), 2.97-2.84 (m, 1H) ppm.

The corresponding ¹H NMR spectrum in CD₃OD is shown in FIG. 4, part 3.

HPLC-MS analysis of VIR-1-097-pH1:
Column: C-18-Reversed-Phase column of the type “Poroshell® 120 SB—C18, 3.0×100 mm, 2.7 μm” by Agilent Technologies. Flow: Constant flow rate 0.7 mL/min, T=35° C.
Method: 0.0-0.1 min, isocratic, 2% MeCN (98% H₂O+0.05% TFA); 0.1-10.0 min, linear gradient, 2% to 50% MeCN (98% to 0% H₂O+0.05% TFA); 10.0-10.5 min, linear gradient, 50% to 100% MeCN (0% to 98% H₂O+0.05% TFA); 10.5-12.0 min, isocratic, 100% MeCN; 12.0-12.5 min, linear gradient, 100% to 2% MeCN (0% to 98% H₂O+0.05% TFA); isocratic,12.5-14.0 min, isocratic 2% MeCN (98% H₂O+0.05% TFA). UV detection at 254.8 nm.

The results are shown in FIG. 4, part 4.

Spiking with separated isomer as in the second peak in Example 2 revealed that only this isomer of the hydrochloride salt remained after adjusting the aqueous isomer mixture to pH<1 with 1 M HCl. The results are shown in FIG. 4, part 5. As no free apomorphine was found in the HPLC chromatogram, it can be assumed that this is not caused by decomposition of the other isomer (as in separated peak 1 in Example 1), but instead occurs via an acid mediated transesterification.

### Example 4: Cation Exchange of Apomorphine Monophosphate

Cation exchange chromatography was performed using different forms of Amberlite™ IR-120 ion exchange resin (0.6-0.8 mm particle size, >1.8 eq/L total capacity as Na-form). For this purpose, Amberlite™ IR-120 Na-form ion exchange resin was pre-equilibrated with a saturated aqueous solution of the corresponding chloride or iodide salt of the desired cation (K⁺, NH₄⁺, Ca²⁺, Mg²⁺) for 3 h. The pre-equilibrated resin was then loaded in a column (diameter: 1 cm, height: 10 cm; resin volume: 7.9 mL; total capacity: >14 mmol) and rinsed twice with 25 mL saturated aqueous solution of the corresponding salt. The resin was then thoroughly washed with deionized water (3×50 mL) to remove any adhering salt. Subsequently, 100 mg apomorphine monophosphate (“VIR-1-097”) (0.242 mmol; Na-form) was dissolved in 1 mL H₂O and the solution was loaded on the ion exchange resin and then eluted (eluent: H₂O; fraction size: 2 mL; fraction analysis: HPLC). Fractions containing the desired product were pooled and lyophilized to give the cation exchanged product.

### Apomorphine Monophosphate Potassium Salt (VIR-1-097-K)

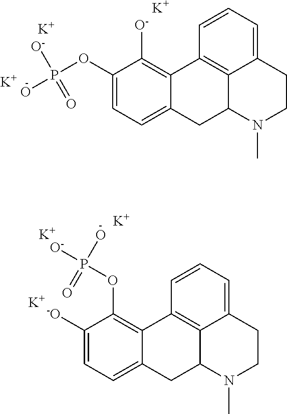

111 mg (0.240 mmol, 99%), ochre solid, C₁₇H1₅K3NO₅P [461.58]
Hygroscopic, highly soluble in water.

### Apomorphine Monophosphate Ammonium Salt (VIR-1-097-NH₄)

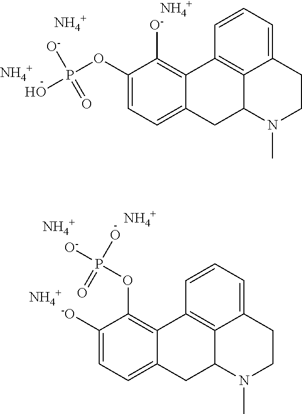

87 mg (0.218 mmol, 90%), brown solid, C₁₇H2₇N40₅P [398.40]
Solubility in water slightly reduced compared to Na-salt

### Apomorphine Monophosphate Calcium Salt (VIR-1-097-Ca)

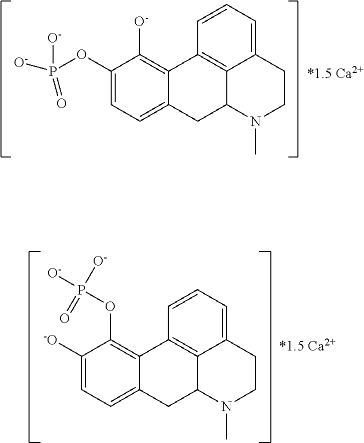

80 mg (0.198 mmol, 82%), grey solid, C₁₇H₁₅Ca_{1.5}NO₅P [404.40]
Reduced solubility in water

### Apomorphine Monophosphate Magnesium Salt (VIR-1-097-Mg)

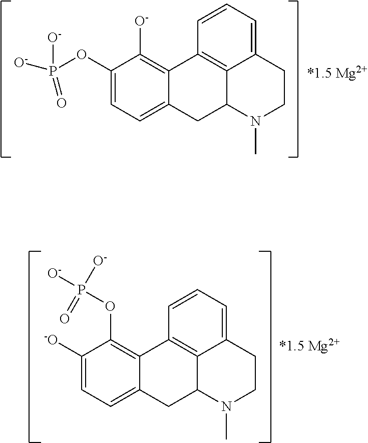

67 mg (0.176 mmol, 73%), off-white solid, C₁₇H₁₅Mg_{1.5}NO₅P [380.74]
Reduced solubility in water

HPLC analysis of the apomorphine monophosphate (VIR-1-097) salts:
Column: C-18-Reversed-Phase column of the type “Poroshell® 120 SB—C18, 3.0×100 mm, 2.7 μm” by Agilent Technologies. Flow: Constant flow rate 0.7 mL/min, T=35° C.
Method: 0.0-0.1 min, isocratic, 2% MeCN (98% H₂O+0.05% TFA); 0.1-8.0 min, linear, 2% to 100% MeCN (98% to 0% H₂O+0.05% TFA); 8.0-11.1 min, isocratic, 100% MeCN; 11.1-11.3 min, linear, 100% to 2% MeCN (0% to 98% H₂O+0.05% TFA); 11.3-12.0 min, isocratic, 2% MeCN (98% H₂O+0.05% TFA). UV detection at 254.8 nm.

The results are shown in FIG. 5.

### Example 5: Preparation of an Injectable Solution of Apomorphine Monophosphate Trisodium Salt

720.0 g of Water for Injection (Wfl) were weighed into a glass bottle and degassed with a flow of nitrogen to remove dissolved oxygen. 400 mg of sodium metabisulfite were added to the compounding vessel under magnetic stirring. After complete dissolving of the antioxidant the pH was measured for information (pH 4.49).

5.62 g of apomorphine monophosphate triisodium salt (isomeric mixture, contains about 5.9% of water) were weighed and added to the compounding mixture under magnetic stirring. A clear, amber colored solution is obtained. The pH was adjusted to 7.50±0.1 with 1M HCl. Then the mixture was filled up to the final weight of 800 g, stirred and the pH checked again (pH 7.62). The compounding vessel was then transferred into a glove box with a controlled atmosphere free of oxygen.
Final concentration: 6.61 g/L (on anhydrous basis) of apomorphine monophosphate trisodium salt corresponding to 0.016 mol/L (MW: 413.25 g/mol)

Glass vials and rubber stoppers were washed with Wfl and sterilized via autoclaving. Sterilized vials and stoppers were transferred into the glove box. The compounding solution was passed through a sterile filter membrane (PALL 25 mm Fluorodyne II PVDF). The filtrate was filled into 20R glass vials in portions of 20.5 ml each, stoppered with rubber stoppers under an oxygen free atmosphere (Oxygen <100 ppm) in the glove box. The stoppered vials were sealed with aluminum crimp-caps.

The filled vials gave the following analytical results:

| Parameter | Test Method | Result |
|---|---|---|
| Appearance | Visual | Clear, amber colored solution |
| Sub- visible Particles | Ph. Eur. 2.9.19 | 10 μm: 4/mL |
| | | 25 μm: 0/mL |
| Visible Particles | Ph. Eur 2.9.20 | Free of visible particles |
| Osmolality | Ph. Eur. 2.2.35 | 74 mOsm/kg |
| pH | Ph. Eur. 2.2.3 | 7.5 |
| Assay | HPLC-UV | Apomorphine monophosphate |
| | | 100.23% Apomorphine <0.38% |

The filled vials are suitable to be used in an animal study for further investigations. They have the same pH, the same content of sulfite (0.5 mg/mL) and are equimolar to a commercial injection solution of apomorphine 5 mg/ml (Dacepton).

A placebo product was prepared in a similar manner but leaving out the apomorphine monophosphate trisodium salt.

### Example 6: In Vitro Test Results of Metabolic Stability of Apomorphine Monophosphate (Isomeric Mixture) on Incubation with Human Hepatocytes

The metabolic conversion of apomorphine monophosphate (isomeric mixture) as obtained in Example 1 into apomorphine was studied in a standardized human hepatocyte assay. Upon incubation with human hepatocytes apomorphine monophosphate is rapidly cleared from the reaction mixture. Within 10 min about 30% is converted to apomorphine; within 60 minutes about 75% auf the apomorphine monophosphate was consumed (see FIG. 6, part 1). The half-life of apomorphine monophosphate in this assay was calculated to be 33 min.

A cell-free control group (negative control) showed no significant formation of apomorphine (see FIG. 6, part 3). This confirms that the observed apomorphine formation is indeed caused by hepatocytes and is not the result of an unspecific degradation during the assay.

Apomorphine was found at all sample points with the highest amount found after 10 min. At this time-point the amount of apomorphine correlates with the disappearance of apomorphine monophosphate. The amount of detected apomorphine decreased thereafter.

It is known that apomorphine is metabolized in the liver and this is also reflected in human liver in vitro stability assays (see FIG. 6, part 4).

The hepatocyte in vitro assay confirms that apomorphine monophosphate as prodrug is rapidly converted into apomorphine. In further in vitro tests it could be shown that human plasma, human whole blood, and human liver S9 fraction do not convert apomorphine monophosphate to apomorphine to any significant extent. This confirms that the release of apomorphine from the apomorphine prodrug is caused by the hepatic metabolism and enzymatic reactions located in functional liver cells.

As a phosphate prodrug the enzymatic activation of apomorphine monophosphate is facilitated by phosphate converting enzymes. One of the most prominent and highly expressed phosphate converting enzymes in the human body is the alkaline phosphatase. High expression levels and abundance of the alkaline phosphatase can be found in human liver cells. Thus, the apomorphine prodrug activation facilitated by the hepatic metabolism and phosphatases, such as the alkaline phosphatase, confirms and rationalizes the in vitro assay results.

### Example 7: Animal Trial—Testing Skin Reaction of the Apomorphine Prodrug of the Present Invention in Pigs

### Materials and Methods

An animal study application for the current in vivo trial was approved by the institutional ethics and welfare committee and the national authority according to Sections 26 et seqq. of the Austrian Animal Experiments Act (Tierversuchsgesetz 2012; approval number 2021-0.746.730).

The aim of this study was to test the skin reaction of two formulations of an apomorphine preparation in pigs: the prodrug of apomorphine according to the present invention and as formulated in Example 5 (APO PD22-03; VIR-1-097, i.e. the sodium salt as described in Example 1), formulated at the concentration of 6.61 mg/mL, at pH 7.5±0.2, further including 0.5 mg/mL metabisulfite, and a commercially available reference apomorphine formulation (Dacepton), which includes apomorphine hydrochloride hemihydrate at the concentration of 5 mg/ml, at pH 3.8±0.2, further including 1.0 mg/mL metabisulfite and 0.8 mg/mL NaCl. The concentration of 6.61 mg/mL of the apomorphine prodrug corresponds to the same molar concentration as the concentration of 5 mg/mL of apomorphine hydrochloride hemihydrate comprised in the Dacepton formulation. Both drugs were administered on the right side of the animals' neck subcutaneously for 12 hours per day over a period of 14 days. In addition, blood was taken via a central venous catheter on selected study days.

For this trial, ten female pigs with an average initial weight of 55 kg were used. After an acclimatization period and daily training through positive conditioning, the six most hand-tame animals and two animals as a backup (for the loss of one animal in the first week) were tattooed on the right side of the neck and a central venous catheter was placed in the jugular vein under general anesthesia.

On the right side of the neck, 8 application fields were tattooed, each divided into 2 quadrants, which determined the location of the needle fixation for the specific day (see FIG. 7). In addition, letters (I-P) were tattooed under the respective application fields. Furthermore, there was a control field in which the needle was inserted and fixed for 12 hours twice at intervals of 7 days, without the catheter and without the application of a drug. The location of insertion of the needle for a specific day was defined by the field in the tattoo. This allowed the assignment of any observed injection site changes to the injection on a specific day. Before inserting the needle, the application field was cleaned of dirt with compresses and disinfected with alcohol.

For the tattooing of the application field and placement of a central venous catheter (into the jugular vein) under general anesthesia, intravenous injection of ketamine hydrochloride (Narketan®, 10 mg/kg body weight) and azaperone (Stresnil®, 1.3 mg/kg body weight) for induction and propofol (2.5-4.0 mg/kg) for maintenance of anesthesia was given and a bolus of methadone (0.3 mg/kg twice every 4 hours) for safe analgesia during the surgery was given to the animals. In order to avoid pain and infections after catheter placement, the animals were given an analgesic (Metacam®, 0.4 mg/kg body weight) and an antibiotic (Baytril RSI®, 7.5 mg/kg body weight) for the first five days after anesthesia. The extension of the central venous catheter was tunneled subcutaneously. The external access was attached to the dorsal part of the neck and to protect this extension, this was protected in a pocket dorsal to the neck fixed with skin sutures.

Since apomorphine is an emetic, an antiemetic was administered perorally to the animals (Motilium 10 mg film-coated tablets, JANSSEN-CILAG Pharma GmbH, 1020 Vienna, Austria) before the start of application of the drug, at midday and shortly before the end of application. The tablet was hidden in a grape and therefore ingested by the animals without any problems.

The medication was applied via a pump application system (D-mine pump; Fa. EVER Neuro Pharma, Unterach, Austria) using a needle for subcutaneous infusion (Neria G27, 6 mm, Unomedical A ConvaTec Company, Denmark). This needle of short length (6 mm) was chosen in order not to penetrate the muscles. The needle was fixed with a tape after fixation (Animal Polster, Fa. Snogg, Vennesla, Norway).

The animals were divided into two groups. Animals numbered 17, 18 and 19 were injected the apomorphine prodrug (APO PD22-03; VIR-1-097) into the right side of the neck. On the other hand, animals 20, 21 and 22 received a commercially available apomorphine formulation (Dacepton) as a control. Since it was no longer possible to collect blood via the central venous catheter on study day 7 from animals 18 and 19 (due to the catheter being clogged), animals 23 and 24 were included in the trial from study day 7 and received the drug APO PD22-03 until study day 14. The same molar concentration was administered for both drugs—in the case of Dacepton 45 mg daily dose=3.7 mg/h=8.88 ml over 12 hours, and a corresponding molar amount of APO PD22-03.

All animals were kept on straw during both the acclimatization and experimental periods and had ad libitum access to pelleted commercial feed and drinking water. The animals were trained to wearing a dog harness throughout the day, which had the pumps enclosed in pockets on the back. During the 14-day trial period, the animals were kept in individual pens, to minimize the risk that animals would scrub each other's harness and/or the fixation of the pumps. To reduce the social stress symptoms, the animals were allowed to have direct nose- and eye-contact at all times.

The study was started with six animals included in the study. In the morning, all animals were clinically examined daily, internal body temperature was measured, and the animals were weighed once a week.

### Macroscopic Evaluation of the Injection Sites

The injection sites were assessed daily before and after the application. The assessed injection site was photo-documented in the morning and in the evening. For evaluation of drug-application derived side-effects (e.g., skin-lesions and noduli formation) the affected skin areas were inspected and observations documented. The size and the extent of the skin lesions were assessed by means of two different scores, i.e., by degree of skin lesion (as: 0—non detectable/present, 1—minimal, 2—low, 3—moderate, 4—high) and by size of the skin lesion (as: 0—no skin reaction detectable, 1-<10% of the field changed, 2-10-25% of the field changed, 3-26-50% of the field changed, 4-51-75% of the field changed, 5-76-100% of the field changed). The occurrence of nodules was counted separately using a score for the number of nodules (0—no nodules detectable; 1—isolated, 1 nodule; 2—low grade, 2-5 nodules; 3—medium grade, 6-10 nodules; 4—high grade, >10 nodules), and the size of the nodules (in centimeters) was assessed.

After the start of the application, the animals were checked every hour to detect a premature departure of the needle. If the needle had not yet come off completely, without fluid leakage, it was allowed to be repaired by reapplication. In case the needle came off completely during the first 8 hours, a new catheter had to be attached, the needle was reinserted into the skin, and the pumps were restarted. If the needle came off after 8 hours, no reapplication was necessary.

The total nodules score per neck, as presented in FIG. 7, is composed of the nodules score (0-4) for each of 14 application sites tested twice a day (the maximum possible score is in this case 56).

### Results

During the experiment, animals 17-24 did not experience any abnormalities during the clinical examination. The three daily applications of the antiemetic worked without any problems and vomiting was not observed in any of the animals. By weighing the animals weekly, a continuous increase in weight and an average daily weight gain within the normal range could be observed. The animals weighed between 50-70 kg at the start of the trial and had between 59-74 kg at the end of the trial (2 weeks later).

### Nodules

Animals 20-22 (Dacepton) showed a nodule after almost every single injection day. However, the average recovery time varied from 5 to 5.14 days in these animals. Animal 20 had nodules for an average of 5.14 days, while animals 21 and 22 had nodules for exactly 5 days on average. Animal 20 had a nodule after 11 of 14 injection days, animal 21 had a total of 12 of 14 possible nodules, and animal 22 had nodules seen after 13 of 14 injection days.

In animals 17-19 (APO PD22-03), significantly fewer nodules were visible. In animal 17, the average recovery time of nodules was 3 days, in animal 18 it was 1 day, and in animal 19 it was 2.5 days. Animal 17 had a total of 3 nodules, animal 18 showed 6 nodules, and animal 19 had only one nodule. Animal 23 and animal 24 started the injection of the drug from study day 7, and both animals showed one nodule each after study day 10, which, however, was seen at one observation time point.

The animals 17-19, which received the formulation APO PD22-03, developed few nodules. In these animals, the nodules were mostly only 1 cm in size at the first observation time point (only animal 19 had a nodule that was 1.5 cm in size at the first observation time point) and had either become smaller (0.5 cm) or disappeared. In animals 20-22, nodules were mostly 1 cm in size at the beginning and remained at 1 cm in diameter. Animal 22 had larger (2 cm) nodules at the first observation time point. Animals 23 and 24 had only one nodule, which was 0.5 cm at one observation time point.

In the control field, where all animals had the needle placed once per week without the application of any drug, no nodules occurred in any animal.

There was a clear difference in the total nodules score between the two drugs. Animals 17-19 had a lower number of nodules per side of the neck. Animal 21 had the highest total score with 15 nodules on the 14th day of injection. The data is summarized in FIG. 7.

### CONCLUSION

These results demonstrate that the formulation comprising the novel apomorphine prodrug according to the present invention exhibits an advantageously improved local tolerability, particularly an advantageously reduced skin reaction, as compared to the commercial apomorphine formulation (Dacepton) used in this animal experiment.

1. A compound of formula (I) or a pharmaceutically acceptable salt thereof: 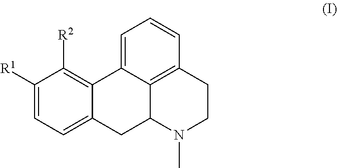
wherein R¹ and R² are each independently —OH or —O—P(═O)(—OH)(—OH), provided that at least one of R¹ and R² is —O—P(═O)(—OH)(—OH).

2. The compound of claim 1, wherein one of R¹ and R² is —O—P(═O)(—OH)(—OH), and the other one of R¹ and R² is —OH.

3. The compound of claim 1, wherein R¹ is —OH and R² is —O—P(═O)(—OH)(—OH).

4. The compound of claim 1, wherein R¹ is —O—P(═O)(—OH)(—OH) and R² is —OH.

5. The compound of claim 1, wherein R¹ and R² are each —O—P(═O)(—OH)(—OH).

6. The compound of any one of claims 1 to 5, wherein said compound has the following configuration:

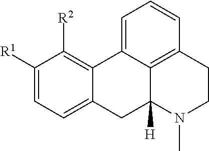

7. The compound of any one of claims 1 to 6, which is a pharmaceutically acceptable salt of the compound of formula (I), preferably a sodium salt.

8. A pharmaceutical composition comprising the compound of any one of claims 1 to 7 and a pharmaceutically acceptable excipient.

9. The compound of any one of claims 1 to 7 or the pharmaceutical composition of claim 8 for use as a medicament.

10. The compound of any one of claims 1 to 7 or the pharmaceutical composition of claim 8 for use in the treatment of a neurodegenerative disease/disorder.

11. The compound for use according to claim 10 or the pharmaceutical composition for use according to claim 10, wherein said neurodegenerative disease/disorder is selected from Parkinson's disease, Alzheimer's disease, Huntington's disease, neuroleptic malignant syndrome, dystonia, and schizophrenia.

12. The compound of any one of claims 1 to 7 or the pharmaceutical composition of claim 8 for use in the treatment of Parkinson's disease.

13. The compound for use according to claim 11 or 12 or the pharmaceutical composition for use according to claim 11 or 12, wherein the compound or the pharmaceutical composition is to be administered in combination with one or more further antiparkinson agents and/or an anti-emetic agent.

14. The compound of any one of claims 1 to 7 or the pharmaceutical composition of claim 8 for use in the treatment of sexual dysfunction or impotence, or for use in the treatment of restless legs syndrome.

15. The compound for use according to any one of claims 9 to 14 or the pharmaceutical composition for use according to any one of claims 9 to 14, wherein the compound or the pharmaceutical composition is to be administered subcutaneously, and/or wherein the compound or the pharmaceutical composition is to be administered to a human subject.
